(19) United States
(12) Patent Application Publication
(10) Pub. No.: US 2022/0000358 A1
(43) Pub. Date: Jan. 6, 2022

(54) PORTABLE EYE IMAGING AND/OR MEASURING APPARATUS

(71) Applicant: Tesseract Health, Inc., Guilford, CT (US)

(72) Inventors: Jacob Coumans, Old Lyme, CT (US); Tyler S. Ralston, Clinton, CT (US); Mark M. Meyers, Madison, CT (US); Maurizio Arienzo, New York, NY (US); Paul E. Glenn, Wellesley, MA (US); Jonathan M. Rothberg, Miami Beach, FL (US); Mohsen Sharifzadeh, Cottonwood Heights, UT (US)

(21) Appl. No.: 17/365,918

(22) Filed: Jul. 1, 2021

Related U.S. Application Data
(60) Provisional application No. 63/155,866, filed on Mar. 3, 2021.
(60) Provisional application No. 63/047,536, filed on Jul. 2, 2020.

(51) Int. Cl. A61B 3/00; A61B 3/10

(52) U.S. Cl. CPC A61B 3/0075; A61B 3/12; A61B 3/102

(57) ABSTRACT

The present disclosure provides improved techniques for imaging and/or measuring a subject's eye. Various aspects of the present disclosure relate to a portable imaging and/or measuring apparatus comprising one or more imaging and/or measuring devices. Some aspects of the present disclosure relate to an imaging and/or measuring device comprising an adjustable flexure having one or more lenses therein. Some aspects of the present disclosure relate to an imaging and/or measuring device comprising an adjustable flexure configured to provide variable diopter compensation. Some aspects of the present disclosure relate to a method comprising imaging and/or measuring a person's eye using an adjustable flexure within an imaging and/or measuring device, the adjustable flexure having one or more lenses therein. Some aspects of the present disclosure relate to a method comprising providing variable diopter compensation for imaging and/or measuring a person's eye using an adjustable flexure.

### CROSS-REFERENCE TO RELATED APPLICATIONS

This application claims the benefit under 35 U.S.C. § 119(e) of: U.S. Provisional Patent Application Ser. No. 63/155,866, filed Mar. 3, 2021, under Attorney Docket No.: T0753.70022US01, and entitled, “PORTABLE EYE IMAGING AND/OR MEASURING APPARATUS;” and U.S. Provisional Patent Application Ser. No. 63/047,536, filed Jul. 2, 2021, under Attorney Docket No.: T0753.70022US00, and titled, “NOVEL FUNDUS IMAGER,” each application of which is hereby incorporated herein by reference in its entirety.

### FIELD OF THE DISCLOSURE

The present disclosure relates to techniques for imaging and/or measuring a subject's eye, including the subject's retina fundus.

### BACKGROUND

Techniques for imaging and/or measuring a subject's eye would benefit from improvement.

### SUMMARY OF THE DISCLOSURE

Some aspects of the present disclosure relate to an imaging and/or measuring device comprising an adjustable flexure having one or more lenses therein.

Some aspects of the present disclosure relate to an imaging and/or measuring device comprising an adjustable flexure configured to provide variable diopter compensation.

Some aspects of the present disclosure relate to a method comprising imaging and/or measuring a person's eye using an adjustable flexure within an imaging and/or measuring device, the adjustable flexure having one or more lenses therein.

Some aspects of the present disclosure relate to a method comprising providing variable diopter compensation for imaging and/or measuring a person's eye using an adjustable flexure.

The foregoing summary is not intended to be limiting. Moreover, various aspects of the present disclosure may be implemented alone or in combination with other aspects.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A is a top perspective view of an exemplary imaging and/or measuring apparatus, according to some embodiments.

FIG. 1B is an exploded view of the imaging and/or measuring apparatus of FIG. 1A, according to some embodiments.

FIG. 1C is a side view of a user operating the imaging and/or measuring apparatus of FIG. 1A, according to some embodiments.

FIG. 1D is a side perspective view of the imaging and/or measuring apparatus of FIG. 1A seated in a stand, according to some embodiments.

FIG. 2 is a top perspective view of an exemplary imaging and/or measuring apparatus having multiple housing portions removed to show white light, fluorescence, optical coherence tomography (OCT), and infrared (IR) imaging and/or measuring components, according to some embodiments.

FIG. 3 is a diagram of exemplary white light and fluorescence components that may be included in the imaging and/or measuring apparatus of FIG. 2, according to some embodiments.

FIG. 4A is a front view of an exemplary white light source that may be included in the white light and fluorescence components of FIG. 3, according to some embodiments.

FIG. 4B is a front view of an exemplary white light and fluorescence excitation source that may be included in the white light and fluorescence components of FIG. 3, according to some embodiments.

FIG. 5A is a front perspective view of exemplary white light and fluorescence components that may be included in the imaging and/or measuring apparatus of FIG. 2, according to some embodiments.

FIG. 5B is an exploded view of the white light and fluorescence components of FIG. 5A, according to some embodiments.

FIG. 6 is a top perspective view of the housing shown in FIGS. 5A-5B with the white light and fluorescence components removed, according to some embodiments.

FIG. 7A is a side perspective view of a diopter flexure of FIGS. 5A-5B, according to some embodiments.

FIG. 7B is a top perspective view of an alternative diopter flexure that may be included in the white light and fluorescence components of FIGS. 5A-5B, according to some embodiments.

FIG. 7C is a side view of the diopter flexure of FIG. 7B, according to some embodiments.

FIG. 7D is a side perspective view of a further alternative diopter flexure that may be included in the white light and fluorescence components of FIGS. 5A-5B, according to some embodiments.

FIG. 8A is a top perspective view of the white light and fluorescence components of FIGS. 5A-5B, according to some embodiments.

FIG. 8B is a side perspective view of the diopter flexure assembly of the white light and fluorescence components of FIGS. 5A-5B, according to some embodiments.

FIG. 8C is a side perspective view of the white light and fluorescence components of FIGS. 5A-5B, according to some embodiments.

FIG. 8D is a rear view of a portion of the white light and fluorescence components of FIGS. 5A-5B, according to some embodiments.

FIG. 8E is a side perspective view of a portion of the white light and fluorescence components of FIGS. 5A-5B, according to some embodiments.

FIG. 9A is a top view of optical paths via the sample and fixation components of the white light and fluorescence components of FIGS. 5A-5B, according to some embodiments.

FIG. 9B is a side perspective view including optical paths of the white light and fluorescence components of FIGS. 5A-5B, according to some embodiments.

FIG. 9C is a top view including optical paths of the white light and fluorescence components of FIGS. 5A-5B, according to some embodiments.

FIG. 10 is a top view of the white light and fluorescence components of FIGS. 5A-5B positioned within an imaging and/or measuring apparatus, according to some embodiments.

FIG. 11 is a top view of an alternative arrangement of white light and fluorescence components that may be included in the imaging and/or measuring apparatus of FIG. 2, according to some embodiments.

FIG. 12 is a top perspective view of a further alternative arrangement of white light and fluorescence components that may be included in the imaging and/or measuring apparatus of FIG. 2, according to some embodiments.

FIG. 13 is a diagram of exemplary OCT and IR components that may be included in the imaging and/or measuring apparatus of FIG. 2, according to some embodiments.

FIG. 14A is a bottom view of source components of the OCT and IR components of FIG. 13, according to some embodiments.

FIG. 14B is a side view of the source components of FIG. 14A, according to some embodiments.

FIG. 15 is a top view of the sample components of the OCT and IR components of FIG. 13, according to some embodiments.

FIG. 16 is a top view of the source and reference components of the OCT and IR components of FIG. 13, according to some embodiments.

FIG. 17A is a top view of the sample and detection components of the OCT and IR components of FIG. 13, according to some embodiments.

FIG. 17B is a side view of the source and detection components of the OCT and IR components of FIG. 13, according to some embodiments.

FIG. 18 is a top view of the sample and fixation components of the OCT and IR components of FIG. 13, according to some embodiments.

FIG. 19 is a top view of the sample and IR components of the OCT and IR components of FIG. 13, according to some embodiments.

FIG. 20A is a top front perspective view of the OCT and IR components of FIG. 13 positioned within an imaging and/or measuring apparatus, according to some embodiments.

FIG. 20B is a top rear perspective view of the imaging and/or measuring apparatus of FIG. 20A, according to some embodiments.

FIG. 21 is a top view of alternative exemplary OCT and IR components within an imaging and/or measuring apparatus, according to some embodiments.

### DETAILED DESCRIPTION

### I. Introduction

The inventors have recognized and appreciated that a person's eyes provide a window into the body that may be used to not only to determine whether the person has an ocular disease, but to determine the general health of the person. The retina fundus in particular can provide valuable information via imaging for use in various health determinations. However, conventional systems of imaging and/or measuring the fundus only provide superficial information about the subject's eye and cannot provide sufficient information to diagnose certain diseases. Accordingly, in some embodiments, multiple modes of imaging and/or measuring are used to more fully image the fundus of a subject. For example, two or more techniques may be used to simultaneously image and/or measure the fundus. In some embodiments, some or each technique of optical imaging, fluorescence imaging, and optical coherence tomography may be used to provide multimodal imaging and/or measuring of the fundus. The inventors have recognized that by using multimodal imaging, as compared to conventional, unimodal imaging, a greater amount of information may be obtained about the fundus than that may be used to determine the health of the subject. In some embodiments, two or more of optical imaging, optical coherence tomography (OCT), fluorescence spectral imaging, and fluorescence lifetime imaging (FLI) may be used to provide multimodal images of the fundus. By way of example, a device that jointly uses color optical imaging, infrared (IR) imaging, OCT, autofluorescence spectral imaging, and FLI provides five modes of imaging the fundus.

The inventors have further recognized and appreciated that making the device portable, handheld, and affordable would have the greatest impact on global health. Countries or regions that cannot afford specialized facilities for diagnosing certain diseases and/or do not have the medical specialists to analyze data from imaging tests are often left behind to the detriment of the overall health of the population. A portable device that may be brought to any low-income community allowing greater access to important healthcare diagnostics. Accordingly, some embodiments are directed to an apparatus that includes multiple modes of imaging the fundus within a housing that is portable and, in some examples, handheld. In some embodiments, the apparatus has a binocular form factor such that a subject may hold the apparatus up to the eyes for fundus imaging. In some embodiments, one or more of the modes of imaging may share optical components to make the apparatus more compact, efficient, and cost effective. For example, a color optical imaging device and a fluorescence imaging device may be housed in a first half of the binocular housing of the apparatus and the OCT device may be housed in the second half of the binocular housing.

Using such an apparatus, both eyes of the subject may be imaged simultaneously using the different devices. For example, the subject's left eye may be imaged using the optical imaging device and/or the fluorescence imaging device while the subject's right eye is imaged using the OCT device. After the initial imaging is complete, the subject can reverse the orientation of the binocular apparatus such that each eye is then measured with the devices disposed in the other half of the binocular housing, e.g., the left eye is imaged using the OCT device and the right eye is imaged using the optical imaging device and/or the fluorescence imaging device. To ensure the apparatus can operate in both orientations, the front surface of the apparatus that is placed near the subject's eyes may be substantially symmetric. Additionally or alternatively, the two halves of the apparatus's housing may be connected by a hinge that allows the two halves to be adjusted to be either orientation.

### II. Exemplary Imaging and/or Measuring Apparatus

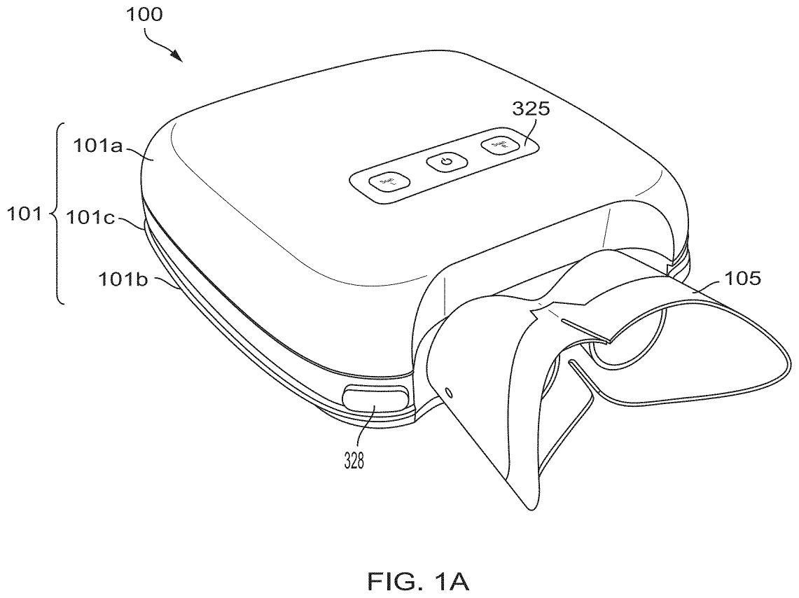

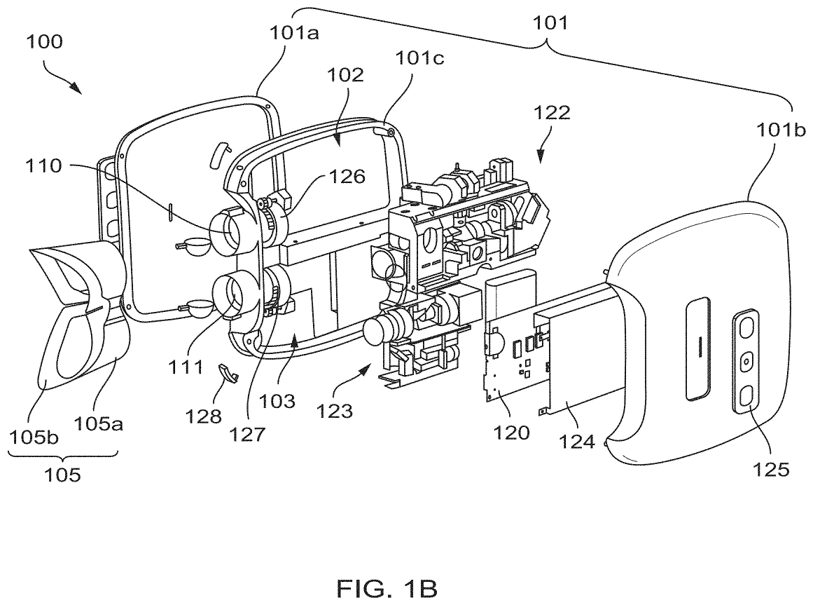

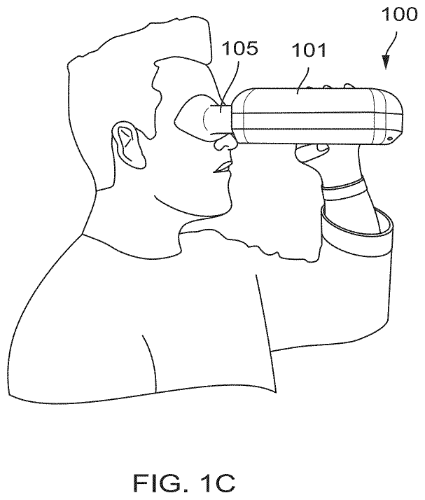

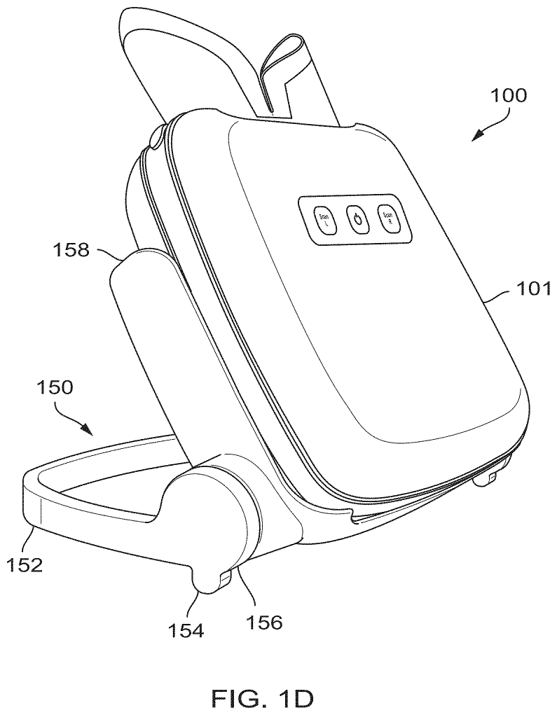

FIGS. 1A-1D illustrate an exemplary embodiment of an imaging (and/or measuring) apparatus 100, according to some embodiments. As shown in FIG. 1A, imaging apparatus 100 has a housing 101, including multiple housing portions 101a, 101b, and 101c. Housing portion 101a has a control panel 125 including multiple buttons for turning imaging apparatus 100 on or off, and for initiating scan sequences. FIG. 1B is an exploded view of imaging apparatus 100 illustrating components disposed within housing 101, such as imaging (and/or measuring) devices 122 and 123 and electronics 120. Imaging devices 122 and 123 may include one or more of: white light imaging components, fluorescence imaging components, infrared (IR) imaging components, and/or OCT imaging components, in accordance with various embodiments. In one example, imaging device 122 may include OCT imaging components and/or IR imaging components, and imaging device 123 may include white light imaging components and/or fluorescence imaging components. In some embodiments, imaging device 122 and/or 123 may include fixation components configured to display a visible fixation object to a user's the imaging apparatus 100. Imaging apparatus 100 further includes front housing portion 105 configured to receive a person's eyes for imaging, as illustrated, for example, in FIG. 1C. FIG. 1D illustrates imaging apparatus 100 seated in stand 150, as described further herein.

As shown in FIGS. 1A-1F, housing portions 101a and 101b may substantially enclose imaging apparatus 100, such as by having all or most of the components of imaging apparatus 100 disposed between housing portions 101a and 101b. Housing portion 101c may be mechanically coupled to housing portions 101a and 101b, such as using one or more screws fastening the housing 101 together. As illustrated in FIG. 1B, housing portion 101c may have multiple housing portions therein, such as housing portions 102 and 103 for accommodating imaging devices 122 and 123. For example, in some embodiments, the housing portions 102 and 103 may be configured to hold imaging devices 122 and 123 in place. Housing portion 101c is further includes a pair of lens portions in which lenses 110 and 111 are disposed. Housing portions 102 and 103 and the lens portions may be configured to hold imaging devices 122 and 123 in alignment with lenses 110 and 111. Housing portions 102 and 103 may accommodate focusing parts 126 and 127 for adjusting the foci of lenses 110 and 111. Some embodiments may further include securing tabs 128. By adjusting (e.g., pressing, pulling, pushing, etc.) securing tabs 128, housing portions 101a, 101b, and/or 101c may be decoupled from one another, such as for access to components of imaging apparatus 100 for maintenance and/or repair purposes.

As shown in FIG. 1B, electronics 120 of imaging apparatus 100 may be configured to perform imaging, measuring, and/or associated processing. In some embodiments, electronics 120 may include one or more processors, such as for analyzing data captured using the imaging devices. In some embodiments, electronics 120 may include wired and/or wireless means of electrically communicating to other devices and/or computers, such as a mobile phone, desktop, laptop, or tablet computer, and/or smart watch. For example, electronics 120 of imaging apparatus 100 may be configured for establishing a wired and/or wireless connection to such devices, such as by USB and/or a suitable wireless network. In some embodiments, housing 101 may include one or more openings to accommodate one or more electrical (e.g., USB) cables. In some embodiments, housing 101 may have one or more antennas disposed thereon for transmitting and/or receiving wireless signals to or from such devices. In some embodiments, imaging devices 122 and/or 123 may be configured for interfacing with the electrical cables and/or antennas. In some embodiments, electronics 120 may be configured to process captured image data based on instructions received from such communicatively coupled devices or computers. In some embodiments, imaging apparatus 100 may initiate an image capture sequence based on instructions received from devices and/or computers communicatively coupled to imaging apparatus 100. In some embodiments, devices and/or computers communicatively coupled to imaging apparatus 100 may process image data captured by imaging apparatus 100. In some embodiments, imaging apparatus 100 may include a battery configured to provide power for operating electronics 120 and imaging devices 122 and 123, such as components of imaging devices 122 and 123 that require power for portable operation (e.g., without being plugged into a power outlet). For example, imaging apparatus 100 may be configured to capture and/or analyze captured images using power supply from the battery, such that imaging apparatus 100 may be portable and configured to capture and process medical grade images using techniques such as white light, FLI, OCT, IR and/or other such techniques, as described further herein.

Control panel 125 may be electrically coupled to electronics 120. For example, the scan buttons of control panel 125 may be configured to communicate an image capture and/or scan command to electronics 120 to initiate a scan using imaging device 122 and/or 123. As another example, the power button of control panel 125 may be configured to communicate a power on or power off command to electronics 120. As illustrated in FIG. 1B, imaging apparatus 100 may further include electromagnetic shielding 124 configured to isolate electronics 120 from sources of electromagnetic interference (EMI) in the surrounding environment of imaging apparatus 100. Including electromagnetic shielding 124 may improve operation (e.g., noise performance) of electronics 120. In some embodiments, electromagnetic shielding 124 may be coupled to one or more processors of electronics 120 to dissipate heat generated in the one or more processors.

As shown in FIG. 1C, for example, during operation of the imaging apparatus 100, a person using the imaging apparatus 100 may place the front housing section 105 against the person's face such that the person's eyes are aligned with the lens portions of imaging apparatus 100. In some embodiments, the imaging apparatus 100 may include a gripping member (not shown) coupled to the housing 101 and configured for gripping by a person's hand. In some embodiments, the gripping member may be formed using a soft plastic material, and may be ergonomically shaped to accommodate the person's fingers. For instance, the person may grasp the gripping member with both hands and place the front housing section 105 against the person's face such that the person's eyes are in alignment with the lens portions.

In some embodiments, imaging apparatus described herein may be configured for mounting to a stand, as illustrated in the example of FIG. 1D. In FIG. 1D, imaging apparatus 100 is supported by stand 150, which includes base 152 and holding portion 158. Base 152 is illustrated including a substantially U-shaped support portion and has multiple feet 154 attached to an underside of the support portion. Base 152 may be configured to support imaging apparatus 100 above a table or desk, such as illustrated in the figure. Holding portion 158 may be shaped to accommodate housing 101 of imaging apparatus 100. For example, an exterior facing side of holding portion 158 may be shaped to conform to housing 101.

As illustrated in FIG. 1D, base 152 may be coupled to holding portion 158 by a hinge 156. Hinge 156 may permit rotation about an axis parallel to a surface supporting base 152. For instance, during operation of imaging apparatus 100 and stand 150, a person may rotate holding portion 158, having imaging apparatus 100 seated therein, to an angle comfortable for the person to image one or both eyes. For example, the person may be seated at a table or desk supporting stand 150. In some embodiments, a person may rotate imaging apparatus 100 about an axis parallel to an optical axis along which imaging devices within imaging apparatus image the person's eye(s). For instance, in some embodiments, stand 150 may alternatively or additionally include a hinge parallel to the optical axis.

In some embodiments, holding portion 158 (or some other portion of stand 150) may include charging hardware configured to transmit power to imaging apparatus 100 through a wired or wireless connection. In one example, the charging hardware in stand 150 may include a power supply coupled to one or a plurality of wireless charging coils, and imaging apparatus 100 may include wireless charging coils configured to receive power from the coils in stand 150. In another example, charging hardware in stand 150 may be coupled to an electrical connector on an exterior facing side of holding portion 158 such that a complementary connector of imaging apparatus 100 interfaces with the connector of stand 150 when imaging apparatus 100 is seated in holding portion 158. In accordance with various embodiments, the wireless charging hardware may include one or more power converters (e.g., AC to DC, DC to DC, etc.) configured to provide an appropriate voltage and current to imaging apparatus 100 for charging. In some embodiments, stand 150 may house at least one rechargeable battery configured to provide the wired or wireless power to imaging apparatus 100. In some embodiments. Stand 150 may include one or more power connectors configured to receive power from a standard wall outlet, such as a single-phase wall outlet.

In some embodiments, front housing portion 105 may include multiple portions 105a and 105b. Portion 105a may be formed using a mechanically resilient material whereas front portion 105b may be formed using a mechanically compliant material, such that front housing portion 105 is comfortable for a user to wear. For example, in some embodiments, portion 105a may be formed using plastic and portion 105b may be formed using rubber or silicone. In other embodiments, front housing portion 105 may be formed using a single mechanically resilient or mechanically compliant material. In some embodiments, portion 105b may be disposed on an exterior side of front housing portion 105, and portion 105a may be disposed within portion 105b.

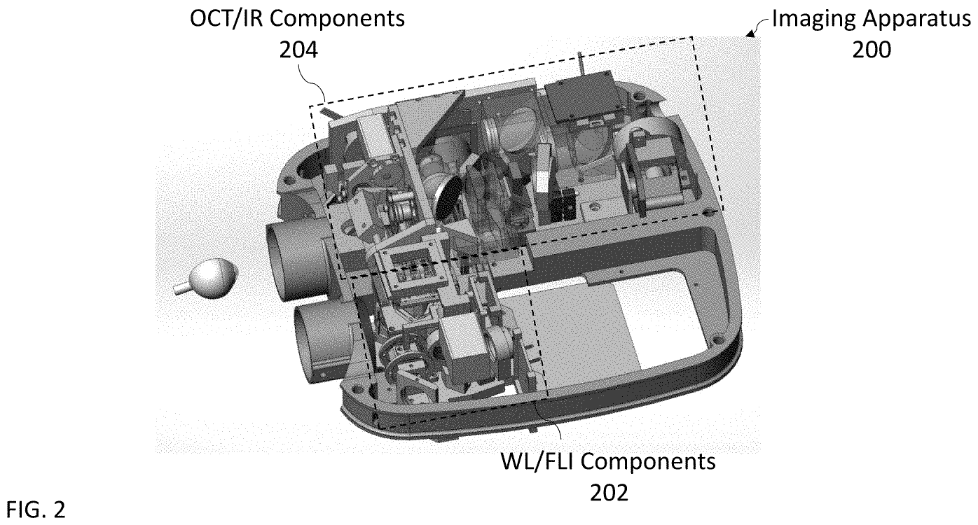

FIG. 2 is a top perspective view of an exemplary imaging and/or measuring apparatus 200 having multiple housing portions removed to show white light and/or fluorescence imaging and/or measuring components 202 and OCT and/or IR imaging and/or measuring components 204, according to some embodiments. As shown in FIG. 2, a first side of imaging and/or measuring apparatus 200 has white light and/or fluorescence components 202 and a second side of imaging and/or measuring apparatus 200 has OCT and/or IR components 204.

In some embodiments, white light components of white light and/or fluorescence components 202 may be configured to illuminate a subject's eye with white light (or a lesser portion of the spectrum of visible light) and receive reflected light from the subject's eye to capture an image of the subject's eye. In some embodiments, fluorescence components of white light and/or fluorescence components 202 may be configured to transmit, to a subject's eye, excitation light configured to excite luminescent molecules in the subject's eye (e.g., naturally luminescent molecules and/or a luminescent dye) and receive fluorescent light from the subject's eye to capture an image of the subject's eye. For example, the fluorescence components may include fluorescence lifetime imaging components, fluorescence intensity imaging components, fluorescence spectral imaging components, and/or a combination thereof.

In some embodiments, OCT components of OCT and/or IR components 204 may be configured to illuminate a subject's eye with light from a light source (e.g., a super-luminescent diode) and compare light reflected from the subject's eye with light reflected from a reference surface to capture an image (e.g., one or more depth scans) of the subject's eye. In some embodiments, IR components of OCT and/or IR components 204 may be configured to illuminate a subject's eye with IR light from an IR light source and receive IR light from the subject's eye to capture an image of the subject's eye.

It should be appreciated that, in some embodiments, white light and/or fluorescence components 202 may include only white light components or only fluorescence components. Similarly, in some embodiments, OCT and/or IR components may include only OCT components or only IR components. Moreover, according to various embodiments, some or each of white light, fluorescence, OCT, and/or IR components may be positioned on either side of an imaging and/or measuring apparatus, alone or in various combinations with one another.

### III. White Light and/or Fluorescence Techniques

Described herein are exemplary configurations of white light and fluorescence imaging and/or measuring components. Although the exemplary configurations illustrated herein include each of white light and fluorescence imaging and/or measuring components, it should be appreciated that white light and/or fluorescence imaging and/or measuring components described herein may be included alone or in combination with one another and/or with other modes of imaging and/or measuring devices.

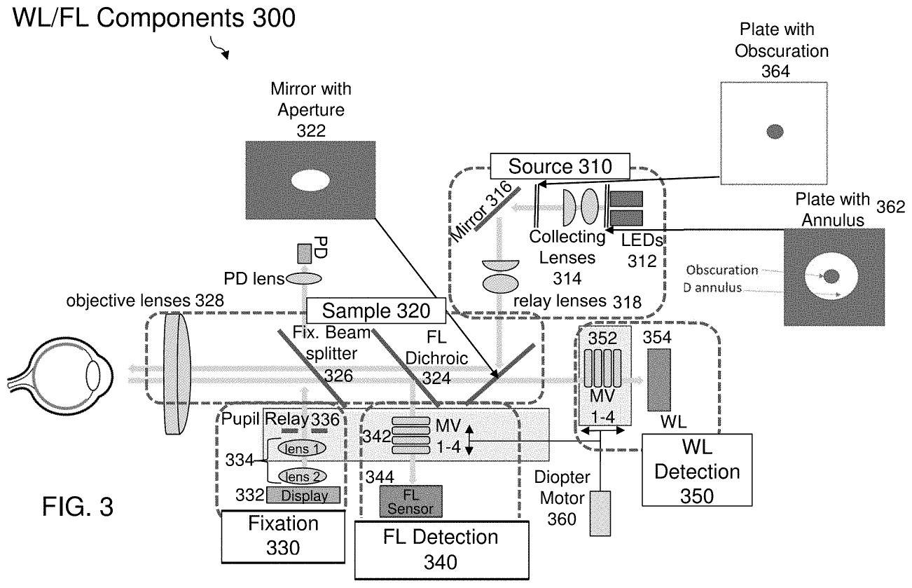

FIG. 3 is a diagram of exemplary white light and fluorescence components 300 that may be included in imaging and/or measuring apparatus 200 (FIG. 2), according to some embodiments. As shown in FIG. 3, white light and fluorescence components 300 include source components 310, sample components 320, fixation components 330, fluorescence detection components 340, and white light detection components 350. In some embodiments, source components 310 may be configured to provide white light and/or excitation light for illuminating and/or exciting luminescent molecules in a subject's eye via sample components 320. In some embodiments, sample components 320 may be configured to receive reflected white light and/or fluorescent light from the subject's eye, provide the received fluorescent light to fluorescence detection components 340 to capture a fluorescence image, and/or provide white light to white light detection components 350 to capture a white light image. In some embodiments, fixation components 330 may be configured to display to the subject's eye via sample components 330 a visible light fixation display. FIG. 3 also shows diopter motor 360, which may be configured to adjust (e.g., focus) machine vision (MV) lenses of fluorescence detection components 340 and/or white light detection components, as described further herein.

In some embodiments, source components 310 may be configured to generate and provide light to sample components 320 for focusing on the subject's eye such that light reflected and/or fluorescence light emitted from the subject's eye may be captured using fluorescence detection components 340 and/or white light detection components 350. In FIG. 3, source components 310 include light emitting diodes (LEDs) 312, collecting lenses 314, mirror 316, and relay lenses 318. In some embodiments, LEDs 312 may include white light LEDs and/or a plurality of color LEDs that combine to substantially cover the visible spectrum, thereby approximating a white light source. For example, in some embodiments, LEDs 312 may be configured to generate light having a wavelength between 400 nanometers (nm) and 700 nm. In some embodiments, LEDs 312 may combine to cover only a portion of the visible spectrum. In some embodiments, LEDs 312 may include one or more blue and/or ultraviolet (UV) lasers configured to excite autofluorescence in the subject's eye.

In some embodiments, LEDs 312 may include one or more fluorescence excitation LEDs, which may be configured to excite luminescent molecules of interest in the subject's eye. In some embodiments, LEDs 312 may be configured to generate excitation light having a wavelength between 460 nm and 500 nm, such as between 480 nm to 500 nm and/or 465 nm to 485 nm. In some embodiments, LEDs 312 may be configured to generate light having a bandwidth of 5-6 nm. In some embodiments, LEDs 312 may be configured to generate light having a bandwidth of 20-30 nm. It should be appreciated that some embodiments may include a plurality of lasers configured to generate light having different wavelengths.

As shown in FIG. 3, source components 300 further include collecting lenses 314, mirror 316, and relay lenses 318. In some embodiments collecting lenses 314 may include one or more collimating lenses and relay lenses 318 may be configured to relay the collimated light to the subject's pupil. In some embodiments, mirror 316 may be configured to direct light from LEDs 312 toward sample components 320.

In FIG. 3, source components 300 further include a plate 362 having an annulus, which may be positioned between LEDs 312 and collecting lenses 314. In some embodiments, plate 362 may be configured to block at least some light from LEDs 312 and transmit at least some light through the annulus. For example, in some embodiments, light transmitted through plate 362 may have a ring shape, such that the illuminated ring may be relayed to the subject's eye. Source components 300 are also shown in FIG. 3 including a plate 364 having an obscuration, which may be positioned between collecting lenses 314 and relay lenses 318. In some embodiments, plate 364 may be configured to block at least some light from reaching a portion of the subject's eye, such as the subject's cornea. The inventors have recognized that the cornea may reflect an undesirably high amount of light that can degrade the quality of images captured targeting other portions of the subject's eye. By blocking at least some light from illuminating the cornea, higher quality images may be obtained.

In some embodiments, sample components 320 may be configured to focus light from source components 310 and fixation light from fixation components 330 on the subject's eye and provide received light (e.g., reflected and/or emitted) from the subject's eye to fluorescence detection components 340 and/or white light detection components 350. In FIG. 3, sample components 320 include mirror 322 having an aperture, fluorescence dichroic 324, fixation beamsplitter 326, and objective lenses 328. In some embodiments, mirror 322 may be configured to receive light from source components 310 and transmit the light to the subject's eye via fluorescence dichroic 324 and fixation beam splitter 326. In some embodiments, the aperture of mirror 322 may be configured to permit light reflected from the subject's eye to reach white light detection components 350. For example, mirror 322 may be configured to block at least some light from the subject's eye from reaching white light detection components 350, such as light reflected from the cornea of the subject's eye.

In some embodiments, fluorescence dichroic 324 may be configured to transmit white light and/or excitation light and reflect fluorescence light such that white light from source components 310 may reach the subject's eye and reflected white light from the subject's eye may reach white light detection components 350, whereas fluorescence dichroic 324 may be configured to reflect fluorescent emissions from the subject's eye toward fluorescence detection components 340. For example, fluorescence dichroic 324 may be configured as a long pass filter. In some embodiments, fluorescence dichroic 324 may be configured to transmit at least some of the received fluorescent emissions to white light detection components 350 and/or reflect at least some of the reflected white light to fluorescence detection components 340. For example, fluorescence dichroic 324 may be configured as a beam splitter for at least some wavelengths of white light and/or fluorescence emissions. According to various embodiments, fluorescence dichroic 324 may have a transmission/reflection transition between 550 nm and 625 nm, such as at 550 nm, 575 nm, 600 nm, or 625 nm.

In some embodiments, fixation beam splitter 326 may be configured to transmit white light, excitation light, and/or fluorescent light and reflect fixation light from fixation components 330 towards the subject's eye, such that white light and excitation light from source components 310 may reach the subject's eye and white light and/or fluorescent light received from the subject's eye may reach white light detection components 350 and/or fluorescence detection components 340, respectively. In some embodiments, fixation beam splitter 326 may be configured as a long pass filter and/or as a beam splitter at least for wavelengths of fixation light. In some embodiments, fixation beam splitter 326 may be configured to transmit light toward a photodetector (PD) and through a PD lens, where the PD is configured to determine whether the amount of light to be transmitted toward the subject's eye exceeds a safety threshold.

In some embodiments, objective lenses 328 may be configured to focus light from source components on the subject's eye and focus light from the subject's eye toward the appropriate detection components. In some embodiments, objective lenses 328 may include a plurality of plano-concave (PCV), plano-convex (PCX), and biconcave lenses. For example, objective lenses 328 may include two opposite-facing PCX lenses with a PCV lens and a biconcave between the PCX lenses. In some embodiments, objective lenses 328 may include an achromatic doublet. For example, the achromatic doublet can include a biconvex (BCX) lens and a meniscus negative lens. In some embodiments, one or more of the lenses of objective lenses 318 can include an aspheric surface, which provides improved image sharpness. For example, the aspheric surface can be a rear surface of the achromatic doublet.

In some embodiments, fixation components 330 may be configured to transmit fixation light toward the subject's eye to display a visible fixation object. In FIG. 3, fixation components 330 include fixation display 332, fixation lenses 334, and pupil relay 336. For example, fixation display 332 may be configured to display a visible fixation object, and fixation lenses 334 may be configured to focus fixation light from fixation display 332 on the subject's eye, such as the subject's pupil via pupil relay 336. In some embodiments, fixation display 332 may be configured to display the fixation object in various positions to cause the subject's eye to move in particular directions when the subject is directed (e.g., by an audio queue from the imaging and/or measuring apparatus and/or a technician) to track the fixation object.

In some embodiments, fluorescence detection components 340 may be configured to receive fluorescent light from the subject's eye reflected via fluorescence dichroic 324. In FIG. 3, fluorescence detection 340 components include machine vision (MV) lenses 342 and fluorescence sensor 344. In some embodiments, MV lenses 342 may be configured to provide diopter compensation for received light from the subject's eye. In some embodiments, MV lenses 342 may be adjustable to provide adjustable diopter compensation. For example, MV lenses 342 may be configured as part of a diopter flexure assembly described further herein. As shown in FIG. 3, MV lenses 342 may be configured to be adjusted by diopter motor 360. For example, diopter motor 360 may be configured to adjust a positioning of MV lenses 342 to adjust the diopter compensation provided by MV lenses 342.

In some embodiments, fluorescence sensor 344 may be configured to capture fluorescent light to perform fluorescence imaging. For example, fluorescence sensor 344 may be an integrated device configured to perform fluorescence lifetime imaging, fluorescence spectral imaging (e.g., autofluorescence spectral imaging), and/or fluorescence intensity imaging. In the example of fluorescence lifetime imaging, fluorescence sensor 344 may be configured to receive incident fluorescent emissions and determine luminance lifetime information of the fluorescent emissions. In the example of fluorescence spectral imaging, fluorescence sensor 344 may be configured to determine luminance wavelength information of the fluorescent emissions. In the example of fluorescence intensity, fluorescence sensor 344 may be configured to determine luminance intensity information of the fluorescent emissions. In some embodiments, fluorescence sensor 344 may have one or more processors integrated thereon, and/or may be coupled to one or more processors onboard the imaging and/or measuring apparatus and configured to provide lifetime, wavelength, and/or intensity information to the processor(s) for image formation and/or measurement.

In some embodiments, fluorescence sensor 344 may be alternatively or additionally configured to capture IR light and perform IR imaging. For example, in some embodiments, an IR light source may be disposed among fluorescence detection components 340 and configured to transmit IR light towards the subject's eye (e.g., by reflection via fluorescence dichroic 324), and fluorescence sensor 344 may be configured to receive reflected IR light from the subject's eye (e.g., by reflection via fluorescence dichroic 324).

In some embodiments, white light detection components 350 may be configured to capture white light received from the subject's eye to produce one or more images and/or measurements of the subject's eye. As shown in FIG. 3, white light detection components 350 may include MV lenses 352 and a white light camera 354. In some embodiments, MV lenses 352 may be configured in the manner described for MV lenses 342. For example, in FIG. 3, MV lenses may be configured to provide adjustable diopter compensation, and diopter motor 360 may be configured to adjust the diopter compensation provided by MV lenses 352 by adjusting a positioning of MV lenses 352. In some embodiments, diopter motor 360 may be configured to adjust MV lenses 342 and 352 independently of one another. For example, diopter motor 360 may be configured to generate motion in two or more orthogonal directions, such as along an axial direction and rotationally about the axial direction, with motion along one direction configured to adjust MV lenses 342 and with motion along another direction configured to adjust MV lenses 352. In some embodiments, diopter motor 360 may be configured to automatically adjust MV lenses 342 and/or 352 based on signals received from one or more processors onboard the imaging and/or measuring apparatus. It should be appreciated that, in some embodiments, diopter motor 360 may be alternatively or additionally configured to adjust MV lenses of fixation components 330.

In some embodiments, white light camera 354 may be configured to produce one or more images and/or measurements of the subject's eye using light received via MV lenses 352. In some embodiments, white light camera 354 may include a color camera. In some embodiments, white light camera 354 may include a monochrome camera. In some embodiments, white light camera 354 may be configured to receive at least some fluorescent emission light via fluorescence dichroic 324. In some embodiments, white light camera 354 may be configured to compensate for differences in spectral power at wavelengths at least partially reflected by fluorescence dichroic 324. In some embodiments white light camera 354 may be configured to output image and/or measurement information to one or more processors onboard the imaging and/or measuring apparatus.

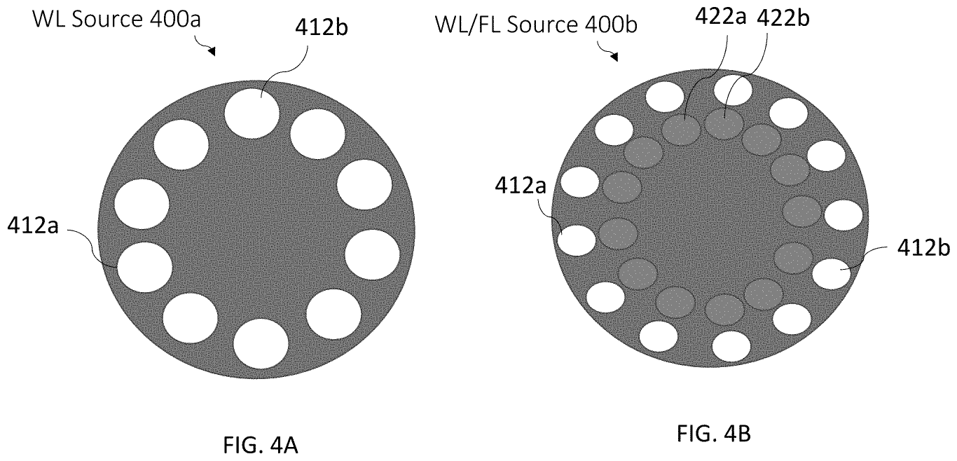

FIG. 4A is a front view of an exemplary white light source 400a that may be included in white light and fluorescence components 300, according to some embodiments. In FIG. 4A, white light source 400a includes white light LEDs arranged in a ring, of which LEDs 412a and 412b are labeled. In some embodiments, the LEDs may be independently controllable, such that only a subset of the LEDs can be illuminated if desired. For example, the LEDs may be configured to illuminate a ring relayed to the subject's eye, and not to illuminate another part of the ring based on turning one or more of the LEDs on while keeping other LEDs off and/or vice versa. For example, the LEDs may be configured not to illuminate undesired areas of the subject's eye, such as the subject's cornea, which may degrade image and/or measurement quality when seeking to capture an image and/or measurement of a different part of the subject's eye. In some embodiments, an excitation light source to be included in white light and fluorescence components 300 may be configured in the manner described for white light source 400a.

FIG. 4B is a front view of an exemplary white light and fluorescence excitation source 400b that may be included in white light and fluorescence components 300, according to some embodiments. As shown in FIG. 4B, white light and fluorescence excitation source 400b includes white light LEDs, of which LEDs 412a and 412b are labeled, and excitation LEDs, of which LEDs 422a and 422b are labeled. In FIG. 4B, the white light LEDs are arranged in a first ring and the excitation LEDs are arranged in a second ring inside the first ring. It should be appreciated that, in some embodiments, the white light LEDs may be arranged in the inner ring and the excitation LEDs may be arranged in the outer ring. In some embodiments, white light and excitation LEDs may be interspersed (e.g., interleaved) in a single ring or in multiple concentric rings. In some embodiments, the white light and/or excitation LEDs shown in FIG. 4B may be independently controllable as described herein for white light source 400a.

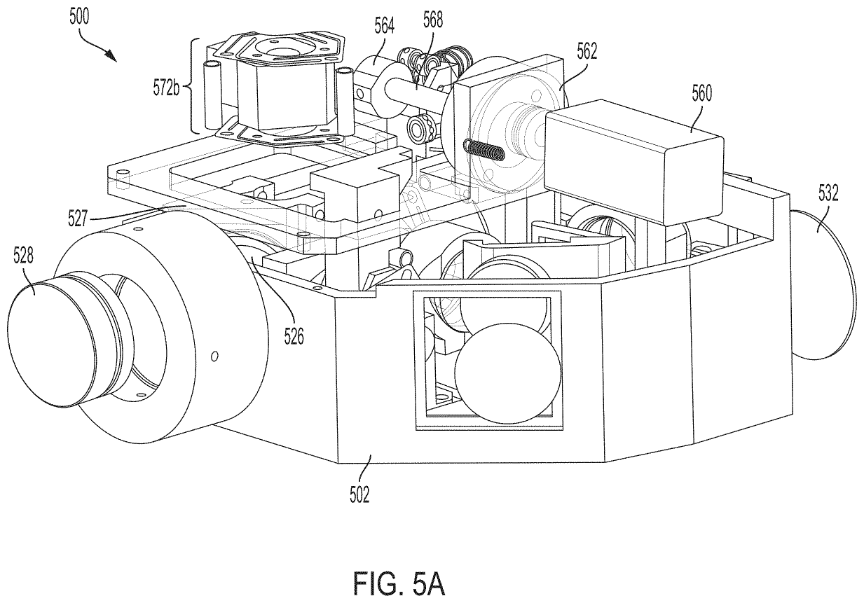

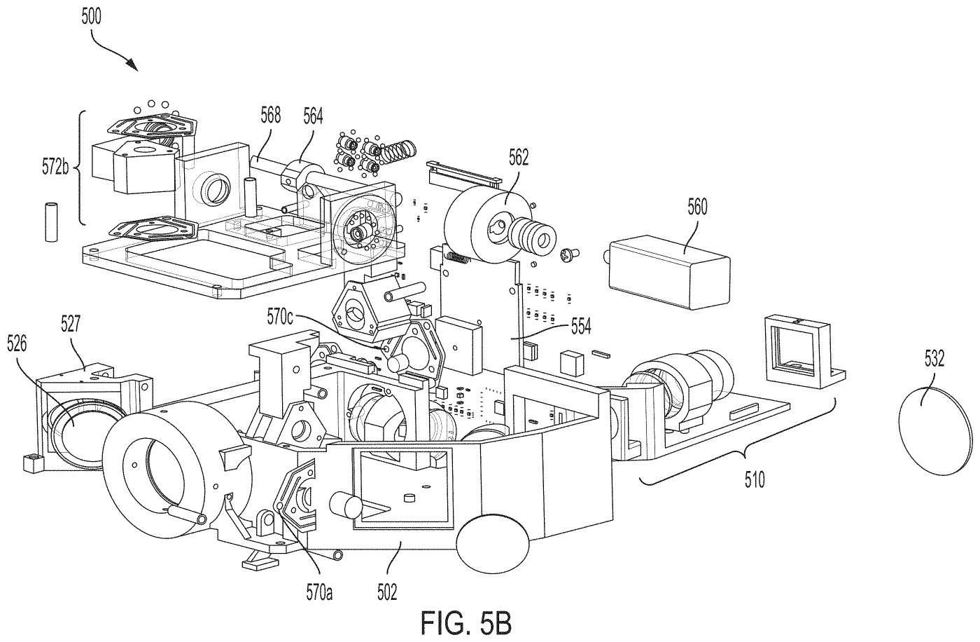

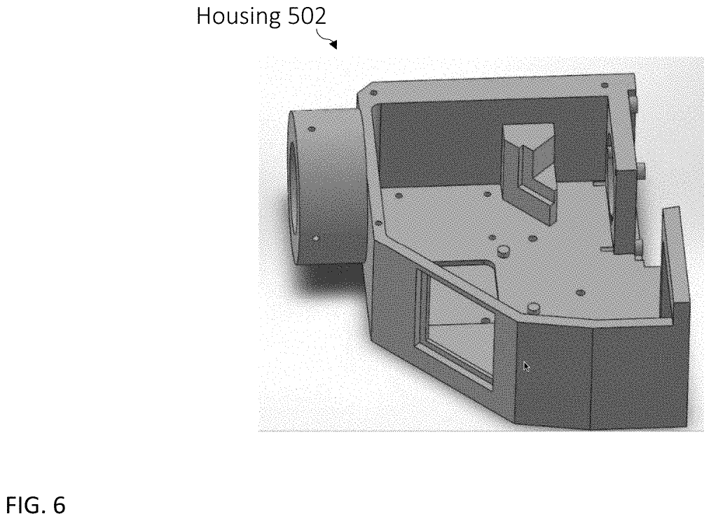

FIG. 5A is a front perspective view of exemplary white light and fluorescence components 500 that may be included in the imaging and/or measuring apparatus of FIG. 2, according to some embodiments. FIG. 5B is an exploded view of white light and fluorescence components 500, according to some embodiments. In FIGS. 5A-5B, white light and fluorescence components 500 are supported by a housing 502. FIG. 6 is a top perspective view of housing 502 with white light and fluorescence components 500 removed, according to some embodiments.

In some embodiments, white light and fluorescence components 500 may be configured in the manner described herein for white light and fluorescence components 300 including in connection with FIGS. 3-4B. For example, in FIGS. 5A-5B, white light and fluorescence components 500 include source components 510, sample components, of which fixation beam splitter 526 and objective lenses 528 are shown in FIG. 5A, fixation components, of which fixation display 532 is shown in FIGS. 5A-5B, and white light detection components, of which white light camera 554 is shown in FIG. 5B. In FIG. 5A, fixation beam splitter 526 is shown supported by a beam splitter housing 527, which may be configured for attaching to housing 502. In FIGS. 5A-5B, white light and fluorescence components 500 also include diopter motor 560 shown mechanically coupled to diopter flexure assemblies 570a and 570c and diopter flexure 572b via cams 562 and 564. Although not shown in FIGS. 5A-5B, it should be appreciated that fluorescence detection components may be optically coupled to the illustrated components via diopter flexure 572b. For example, diopter motor 560 may be configured to adjust diopter flexure assembly 570a to provide variable diopter compensation for the fixation components, diopter flexure 572b to provide variable diopter compensation for the fluorescence detection components, and diopter flexure assembly 570c to provide variable diopter compensation for the white light detection components. It should be appreciated that some embodiments may only include any number of diopter flexures and assemblies as shown in FIGS. 5A-5B.

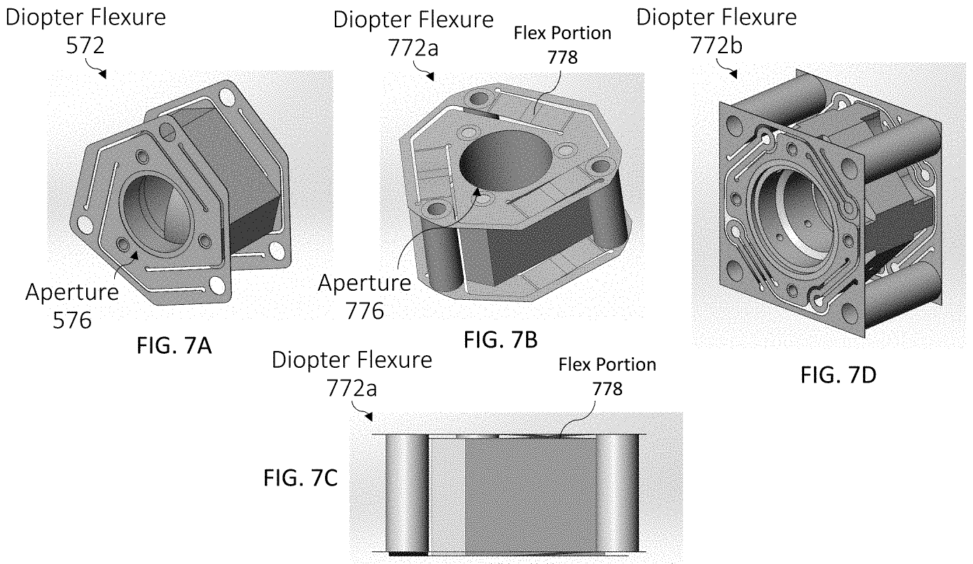

FIG. 7A is a side perspective view of a diopter flexure 572, which may be included as diopter flexure 572b and/or in diopter flexure assemblies 570a and 570b, according to some embodiments. As shown in FIG. 7A, diopter flexure 572 includes aperture 576, which may be configured to support multiple MV lenses spaced from one another in an axial direction through aperture 576. For example, diopter flexure 572 may be configured to compress along the axial direction to adjust a positioning of the MV lenses.

FIG. 7B is a top perspective view of an alternative diopter flexure 772a that may be included in white light and fluorescence components 500, according to some embodiments. FIG. 7C is a side view of diopter flexure 772a, according to some embodiments. In some embodiments, diopter flexure 572 may be configured to operate in the manner described herein for diopter flexure 772a.

In some embodiments, diopter flexure 772a may be configured to compress and/or decompress in response to a force exerted on diopter flexure 772a to adjust a positioning of MV lenses positioned along the axial direction through aperture 776. As shown in FIGS. 7B-7C, diopter flexure 772a includes flex portions 778 mechanically coupling the interior portion of diopter flexure 772a, which includes aperture 576, to the exterior portion of diopter flexure 772a. For example, flex portions 778 may be configured to allow the interior portion of diopter flexure 772a to compress relative to the exterior portion when a force is exerted on the interior portion along the axial direction (e.g., the direction through aperture 576) and to decompress relative to the exterior portion when the force is no longer exerted. FIG. 7C shows diopter flexure 772a in the decompressed state, with a gap shown along the axial direction between the exterior and interior portions of diopter flexure 772a mechanically coupled to one another via flex portions 778. In some embodiments, when compressed, substantially no gap exists along the axial direction between the interior and exterior portions of diopter flexure 772a.

FIG. 7D is a side perspective view of a further alternative diopter flexure 772b that may be included in white light and fluorescence components 500, according to some embodiments. In some embodiments, diopter flexure 772b may be configured in the manner described for diopter flexures 572 and 772a.

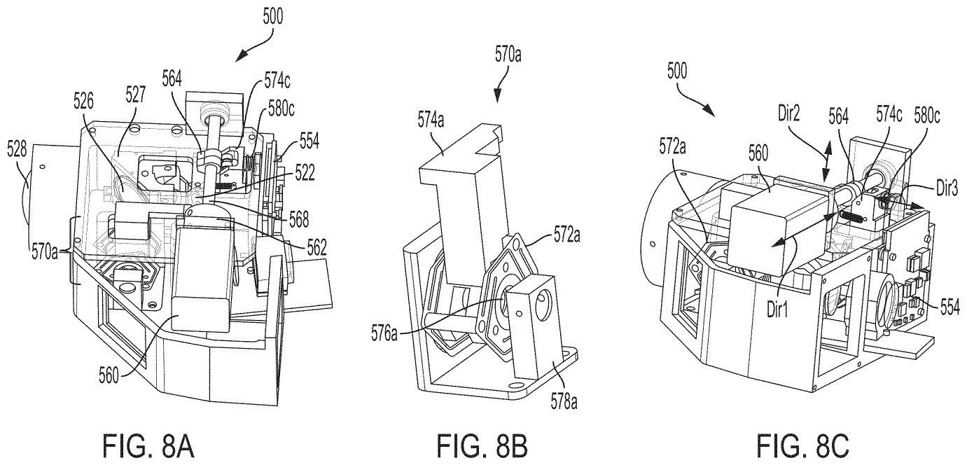

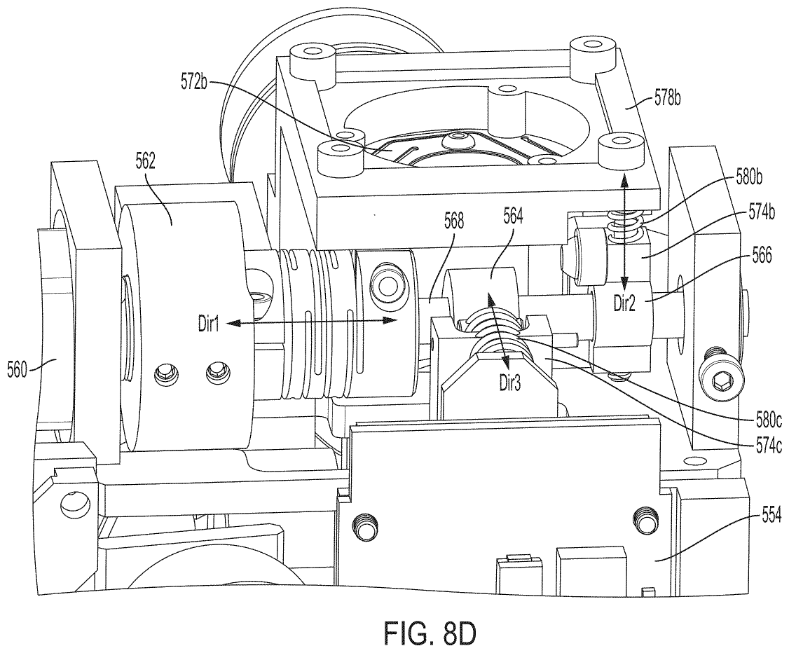

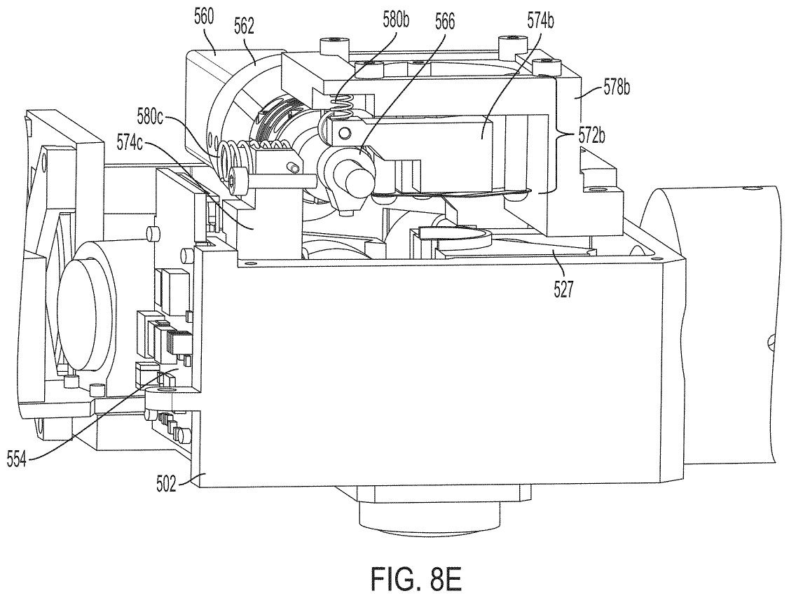

FIG. 8A is a top perspective view further illustrating white light and fluorescence components 500, according to some embodiments. FIG. 8B is a side perspective view of diopter flexure assembly 570a of white light and fluorescence components 500, according to some embodiments. FIG. 8C is a side perspective view of the white light and fluorescence components of FIGS. 5A-5B, according to some embodiments. FIG. 8D is a rear view of a portion of the white light and fluorescence components of FIGS. 5A-5B, according to some embodiments. FIG. 8E is a side perspective view of a portion of the white light and fluorescence components of FIGS. 5A-5B, according to some embodiments.

As shown in FIG. 8B, diopter flexure assembly 570a includes diopter flexure 572a supported by flexure housing 578a and mechanically coupled to flexure lever 574a. In some embodiments, diopter flexure 572a may be configured in the manner described herein for diopter flexure 572 including in connection with FIG. 7A. In some embodiments, flexure housing 578a may be configured to mount diopter flexure 572a to housing 502. In some embodiments, flexure lever 574a may be configured to engage a cam (e.g., cam 564) to mechanically couple diopter flexure 572a to diopter motor 560, as described further herein. In some embodiments, diopter flexure assemblies 570b and 570c may be configured in the manner described herein for diopter flexure assembly 570a, as described further herein.

As shown in FIGS. 8A and 8C, diopter motor 560 may be mechanically coupled to diopter flexure assembly 570a via cam 562. For example, diopter motor 560 is shown in FIGS. 8A and 8C as part of a diopter motor assembly that includes camshaft 568 with cams 562, 564, and 566 disposed along camshaft 568. In some embodiments, diopter motor 560 may be configured to move cam 562 in a first direction Dir1, which may be parallel to the axial direction through aperture 576a of the diopter flexure 572a. For example, diopter motor 560 may be configured to displace camshaft 568 along the first direction Dir1. In this example, diopter motor 560 may be configured to push cam 562 against flexure lever 574a along the first direction Dir1, thereby compressing diopter flexure 572a along the first direction Dir1 to reposition the MV lenses of diopter flexure 572a, thereby providing variable diopter compensation for the fixation components. In some embodiments, diopter flexure assembly 570a may include a spring connected between housing portions of the assembly. For example, the spring may be configured to decompress diopter flexure 572a along the first direction Dir1, such that the spring decompresses diopter flexure 572a unless cam 562 compresses diopter flexure 572a against the force of the spring. In the example of FIG. 8B, such a spring may be connected between flexure lever 574a and a portion of flexure housing 578a.

In some embodiments, diopter motor 560 may be mechanically coupled to diopter flexure 572b to provide variable diopter compensation for the fluorescence detection components. For example, as shown in FIGS. 8D and 8E, diopter motor 560 may be configured to rotate cam 566 about the first direction Dir1, such as by rotating camshaft 568 about the first direction Dir1, such that cam 566 presses against flexure lever 574b along a second direction Dir2 parallel to the axial direction through the aperture of diopter flexure 572b. For instance, cam 566 may be thereby configured to compress diopter flexure 572b along the second direction Dir2. In this example, as shown in FIG. 8D, spring 580b may be connected between flexure lever 574b and flexure housing 578b and configured to decompress diopter flexure 572b along the second direction Dir2, such that spring 580b decompresses diopter flexure 572b unless cam 566 presses flexure lever 574b against the force of spring 580b. Also in this example, diopter motor 560, via cam 566, may be configured to apply a force at least partially along the second direction Dir2. For example, cam 566 may be configured to apply a rotational force about the first direction Dir1 that translates, at least in part, along the second direction Dir2 when pressed against flexure lever 574b.

In some embodiments, diopter motor 560 may be mechanically coupled to diopter flexure 572c to provide variable diopter compensation for the white light detection components via cam 564, such as by rotating cam 564 about the first direction Dir1. For example, as shown in FIGS. 8D and 8E, diopter motor 860 may be configured to rotate cam 564 about the first direction Dir1, such as by rotating camshaft 568 about the first direction Dir1, such that cam 564 presses against flexure lever 574c along a third direction Dir3 parallel to the axial direction through the aperture of diopter flexure 572c. For instance, cam 564 may be thereby configured to compress diopter flexure 572c along the third direction Dir3. In this example, as shown in FIGS. 8A and 8C-E, spring 580c may be connected between flexure lever 574c and flexure housing 578c (and/or between flexure lever 574c and housing 502) and configured to decompress diopter flexure 572c unless cam 564 presses flexure lever 574c against the force of spring 580b. Also in this example, diopter motor 560, via cam 564, may be configured to apply a force at least partially along the third direction Dir3. For example, cam 566 may be configured to apply a rotational force about the first direction Dir1 that translates, at least in part, along the third direction Dir3 when pressed against flexure lever 574c.

In some embodiments, diopter motor 560 may be configured to adjust diopter flexure 572a, 572b, and/or 572c with precision that can provide high resolution diopter compensation for the white light and/or fluorescence components 500. For example, diopter motor 560 may be configured to adjust diopter flexure 572a, 572b, and/or 572c with a precision of less than 100 micrometers and/or a precision of less than 50 micrometers. In this example, the precision with which diopter motor 560 is configured to adjust diopter flexures can correspond to movement distances from diopter to diopter of the lenses of the diopter flexures.

It should be appreciated that some embodiments may include multiple diopter motors, as embodiments described herein are not so limited. For example, a separate diopter motor may be coupled to each diopter flexure assembly 570a, 570b, and/or 570c.

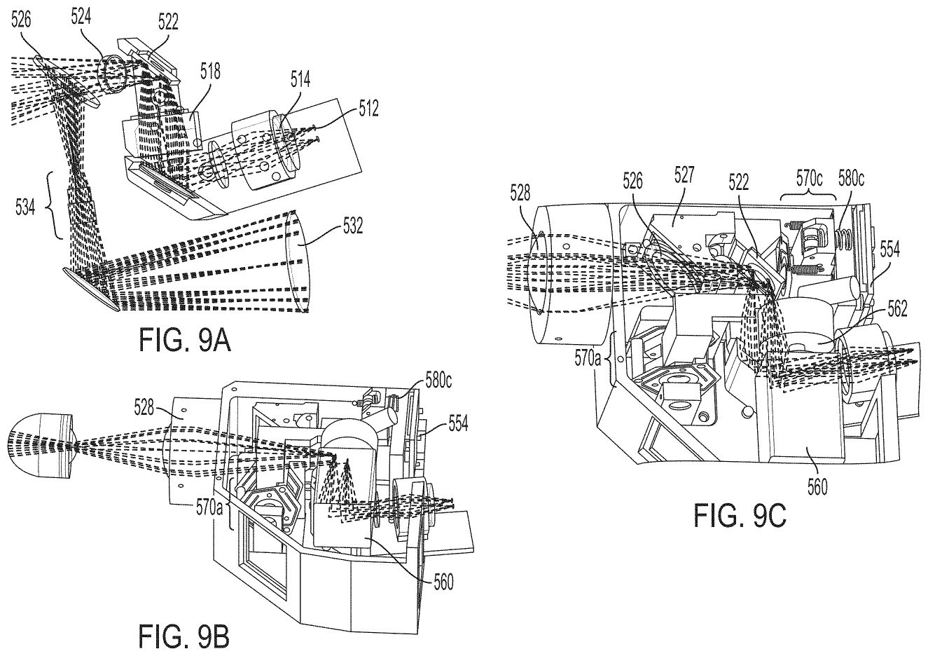

FIG. 9A is a top view of optical paths via the sample and fixation components of white light and fluorescence components 500, according to some embodiments. In FIG. 9A, the housing 502 has been removed, and LEDs 512, collecting lenses 514, relay lenses 518, mirror 522, and fluorescence dichroic 524 are shown to illustrate the path of white light and/or excitation light toward the subject's eye. FIG. 9A also shows the path of fixation light from fixation display 532 via fixation lenses 534 (e.g., MV lenses within diopter flexure 572a) to the subject's eye.

FIG. 9B is a side perspective view including optical paths of white light and fluorescence components 500, according to some embodiments. FIG. 9C is a top view including optical paths of white light and fluorescence components 500, according to some embodiments. FIGS. 9B-9C further illustrate the path of white light and/or excitation light through objective lenses 528 toward the subject's eye.

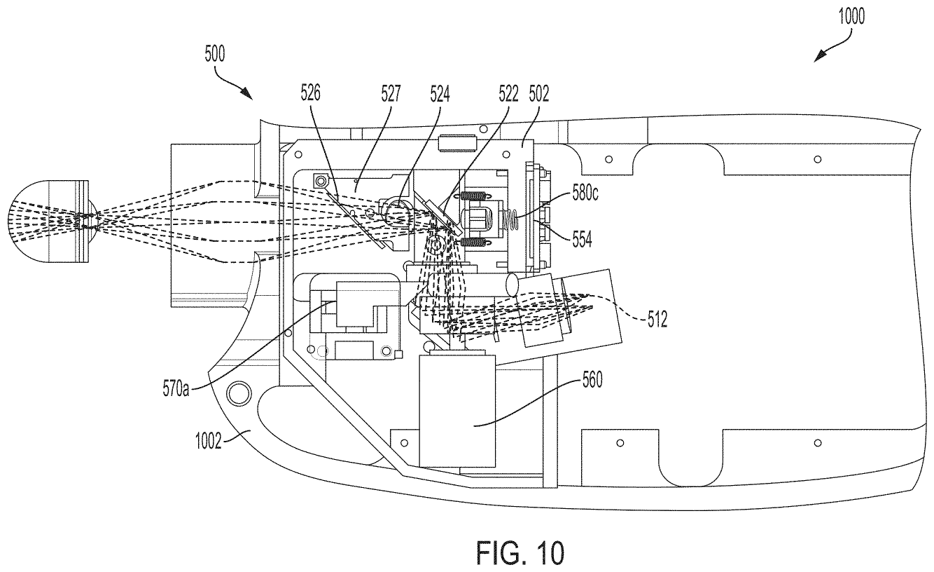

FIG. 10 is a top view of white light and fluorescence components 500 positioned within imaging and/or measuring apparatus 1000, according to some embodiments. As shown in FIG. 10, housing 502 supporting white light and fluorescence components 500 is positioned within a housing 1002 of imaging and/or measuring apparatus 1000. In some embodiments, imaging and/or measuring apparatus 1000 may further include OCT and/or IR components.

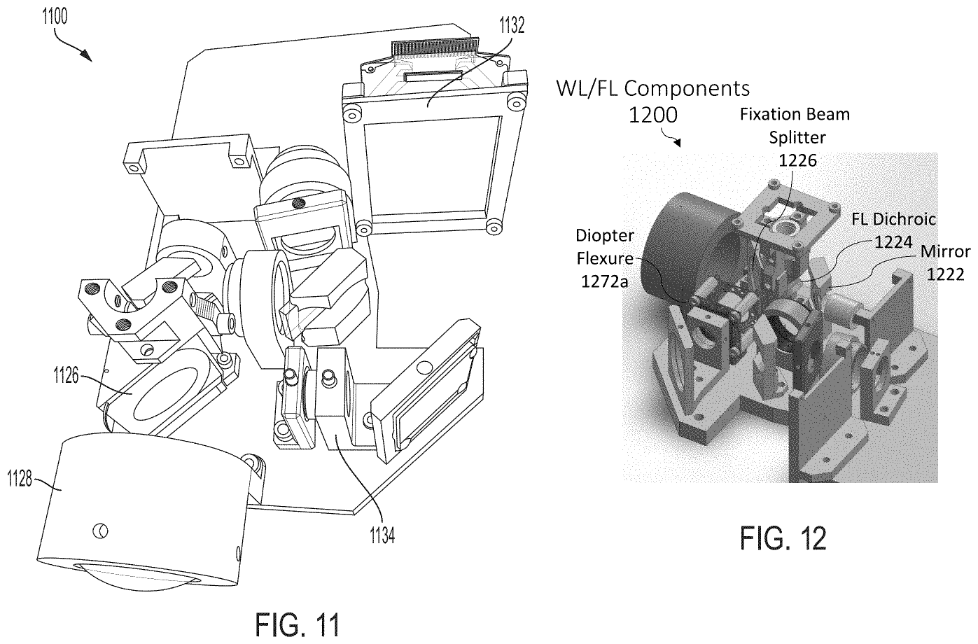

FIG. 11 is a top view of an alternative arrangement of white light and fluorescence components 1100 that may be included in imaging and/or measuring apparatus 200, according to some embodiments. In some embodiments, white light and fluorescence components 1100 may be configured in the manner described herein for white light and fluorescence components 500 including in connection with FIGS. 5A-10. For example, in FIG. 11, white light and fluorescence components 1100 include fixation beam splitter 1126, objective lenses 1128, fixation display 1132, and fixation lenses 1134. Although not shown in FIG. 11, it should be appreciated that white light and fluorescence components 1100 may include white light and/or excitation source components, sample components, white light detection components, and fluorescence detection components as described herein for white light and fluorescence components 500. Alternatively or additionally, in some embodiments, white light and fluorescence components 1100 may include one or more diopter motors and one or more diopter flexures as described herein for white light and fluorescence components 500.

FIG. 12 is a top perspective view of a further alternative arrangement of white light and fluorescence components 1200 that may be included in the imaging and/or measuring apparatus of FIG. 2, according to some embodiments. In some embodiments, white light and fluorescence components 1200 may be configured in the manner described herein for white light and fluorescence components 500 and/or 1100. For example, as shown in FIG. 12, white light and fluorescence components 1100 include mirror 1222, fluorescence dichroic 1224, fixation beam splitter 1226, and diopter flexure 1272a. Although not shown in FIG. 12, it should be appreciated that white light and fluorescence components 1200 may include white light and/or excitation source components, fixation components, white light detection components, and fluorescence detection components as described herein for white light and fluorescence components 500 and 1100. Alternatively or additionally, in some embodiments, white light and fluorescence components 1100 may include one or more diopter motors and one or more additional or alternative diopter flexures as described herein for white light and fluorescence components 500 and 1100.

### IV. Optical Coherence Tomography and/or Infrared Techniques

Described herein are exemplary OCT and IR imaging and/or measuring components. Although the exemplary configurations described herein include each of OCT and IR imaging and/or measuring components, it should be appreciated that OCT and/or IR imaging and/or measuring components described herein may be included alone or in combination with one another and/or with other modes of imaging and/or measuring devices.

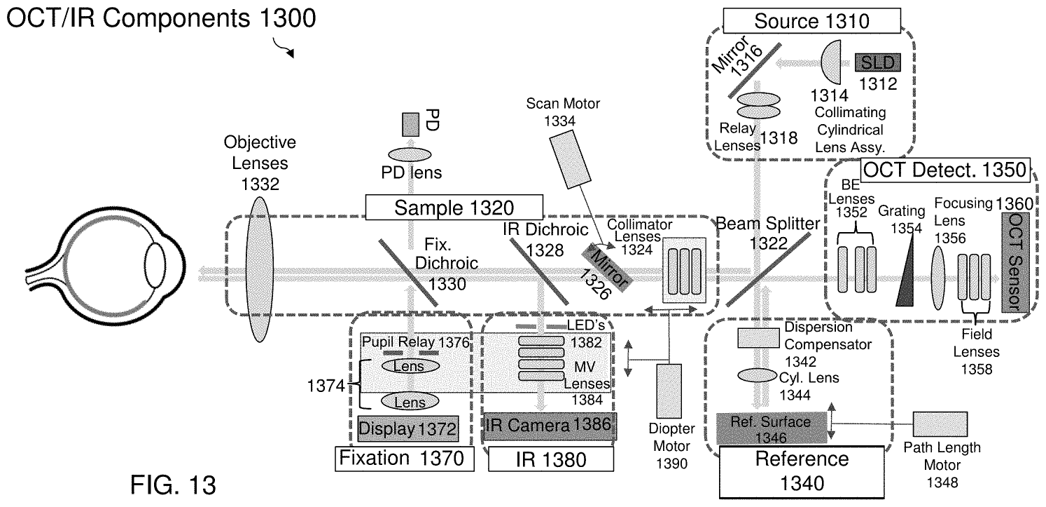

FIG. 13 is a diagram of exemplary OCT and IR components 1300 that may be included in the imaging and/or measuring apparatus of FIG. 2, according to some embodiments. In FIG. 13, OCT and IR components 1300 include source components 1310, sample components 1320, reference components 1340, OCT detection components 1350, fixation components 1370, and IR components 1380. In some embodiments, source components 1310 may be configured to illuminate a subject's eye with light via sample components 1320 and illuminate a reference surface 1346 of reference components 1340 to provide sample light and reference light to OCT detection components 1350 for capturing one or more OCT depth scans. In some embodiments, fixation components 1370 may be configured to display to the subject's eye a visible fixation target as described herein for fixation components 330 including in connection with FIGS. 3-12. In some embodiments, IR components may be configured to receive IR light from the subject's eye via sample components 1320 and capture an IR image and/or measurement of the subject's eye using the received IR light. As shown in FIG. 13, source components 1310, sample components 1320, reference components 1340, and OCT detection components 1350 may be coupled to one another via beam splitter 1322. Also shown in FIG. 13, OCT and IR components 1300 include diopter motor 1390, which may be configured to adjust lenses of sample components 1320, fixation components 1370, and/or IR components 1380 in the manner described herein for diopter motor 360 including in connection with FIGS. 3-12.

In some embodiments, source components 1310 may be configured to provide source light for illuminating the subject's eye via sample components 1320 and illuminating a reference surface 1346 of reference components 1340. In FIG. 13, source components 1310 include a superluminescent diode (SLD) 1312, collimating cylindrical lens assembly 1314, mirror 1316, and relay lenses 1318. In some embodiments, SLD 1312 may be configured to provide broadband light, such as including white light and IR light. In some embodiments, SLD 1312 may be configured to provide light over a spectral width greater than 40 nm. In some embodiments, SLD 1312 may be configured to provide light having a center wavelength between 750 nm and 900 nm. For example, SLD 1312 may be configured to provide light having a center wavelength of 850 nm. In some instances, there may be scattering by the tissue of the subject's eye at 850 nm than at other wavelengths. In some embodiments, SLD 1312 may be configured to provide polarized light (e.g., linearly, circularly, or elliptically polarized). In some embodiments, SLD 1312 may be configured to have a single lateral spatial mode.

In some embodiments, source components 1310 may alternatively or additionally include a vertical-cavity surface-emitting laser (VCSEL) with an adjustable mirror on one side. In some embodiments, the VCSEL may have a wavelength tuning range of greater than 100 nm using a micro-mechanical movement (MEMs). In some embodiments, source components 1310 may alternatively or additionally include a plurality of light sources that combine to achieve broad light spectral width, such as including a plurality of laser diodes, which can be a cost-effective way of achieving higher brightness and shorter pulse duration than SLDs in some cases.

In some embodiments, collimating cylindrical lens assembly 1314 may be configured to collimate light from SLD 1312 for illuminating the subject's eye. In some embodiments, source components 1310 may be configured to illuminate a line across the subject's eye to simultaneously perform a plurality of depth scans of the subject's eye. For example, collimating cylindrical lens assembly 1314 may be configured to transmit light from SLD 1312 in a line to illuminate the line across the subject's eye. In some embodiments, mirror 1316 may be configured to reflect the collimated light toward relay lenses 1318, which may be configured to relay the collimated light toward the subject's eye (e.g., pupil) via sample components 1320.

As shown in FIG. 13, source components 1310 are coupled to sample components 1320 and reference components 1340 via beam splitter 1322. In some embodiments, beam splitter 1322 may be configured to reflect light from source components 1310 toward the subject's eye, transmit light from source components 1310 toward reference components 1340, transmit light received from the subject's eye toward OCT detection components 1350, and reflect light received from reference components 1340 toward OCT detection components 1350. For example, beam splitter 1322 may be configured as a 50/50 beam splitter, such as transmitting 50% of light from source components 1310 toward sample components 1320 and 50% of the light toward reference components 1340.

In some embodiments, sample components 1320 may be configured to provide light from source components 1320 and fixation components 1370 to the subject's eye and provide light received from the subject's eye to OCT detection components 1350 and IR components 1380. In FIG. 13, sample components 1320 include collimator lenses 1324, scan mirror 1326, IR dichroic 1328, fixation dichroic 1330, objective lenses 1332, and scan motor 1334.

In some embodiments, collimator lenses 1324 may be configured to provide a variable collimation of light from beam splitter 1322 toward the subject's eye via scan mirror 1326. For example, diopter motor 1390 may be configured to adjust a positioning of collimator lenses 1324 to adjust the collimation provided by collimator lenses 1324. In some embodiments, scan motor 1334 may be configured to steer scan mirror 1326 to steer light from beam splitter 1322 toward different portions of the subject's eye. For example, in some embodiments, source components 1310 may be configured to provide a line of illumination to scan mirror 1326, and scan mirror 1326 may be configured to steer the line of illumination across the subject's eye in a direction perpendicular to the line of illumination and perpendicular to the depth of the subject's eye. In some embodiments, the line of illumination may be horizontal across the subject's eye and scan mirror 1326 may be configured to steer the line of illumination vertically. It should be appreciated that any pair of perpendicular directions that are perpendicular to the depth direction of the subject's eye may be used for the illumination line and for steering using scan mirror 1326. In some embodiments, scan motor 1334 and/or scan mirror 1326 may include one or more stepper motors, galvanometers, polygonal scanners, microelectromechanical switch (MEMS) mirrors, and/or other moving mirror devices.

In some embodiments, IR dichroic 1328 may be configured to transmit light from beam splitter 1322 toward the subject's eye. In some embodiments, IR components 1380 may be configured to transmit IR illumination light to IR dichroic 1328, which may be configured to reflect the IR illumination light toward the subject's eye and reflect received IR light toward IR components 1380 for capturing an IR image and/or measurement of the subject's eye. In some embodiments, IR dichroic 1328 may be configured as a short-pass dichroic. In some embodiments, fixation dichroic 1330 may be configured to transmit light from beam splitter 1322 toward the subject's eye and reflect fixation light from fixation components 1370 toward the subject's eye. In some embodiments, fixation dichroic 1330 may be configured as a long-pass dichroic. In some embodiments, fixation dichroic 1330 may be configured to transmit at least some illumination and/or fixation light toward a PD, where the PD is configured to determine whether the amount of light to be transmitted toward the subject's eye exceeds a safety threshold.

In some embodiments, objective lenses 1332 may be configured to focus illumination light on the subject's eye and focus light received from the subject's eye such that the received light can be captured using OCT detection components 1350 and/or IR components 1380. In some embodiments, objective lenses 1332 may be configured in the manner described herein for objective lenses 328 including in connection with FIGS. 3-12. For example, objective lenses 1332 may include one or more PCV, PCX, and/or biconcave lenses.

In some embodiments, reference components 1340 may be configured to receive illumination light from source components 1310 via beam splitter 1322 and provide light reflected from reference surface 1346 to beam splitter 1322 to reflect toward OCT detection components 1350. In FIG. 13, reference components 1340 include dispersion compensator 1342, cylindrical collimating lens 1344, reference surface 1346 and path length motor 1348. In some embodiments, reference surface 1346 may be configured to receive the illumination light from beam splitter 1322 via dispersion compensator 1342 and cylindrical collimating lens 1344 and reflect light toward beam splitter 1322 via dispersion compensator 1342 and cylindrical collimating lens 1344. In some embodiments, path length motor 1348 may be configured to move reference surface 1346 along the optical path from beam splitter 1322 to adjust a distance traveled by the light from beam splitter 1322 and reflected by reference surface 1346. For example, path length motor 1348 may be configured to position reference surface 1346 within a threshold distance of a distance from beam splitter 1322 to the subject's eye, the latter of which may be determined prior to and/or during OCT imaging and/or measurement.

In some embodiments, OCT detection components 1350 may be configured to receive light from the subject's eye and from reference components 1340 and capture an OCT image and/or measurement using the received light. In FIG. 13, OCT detection components 1350 include beam expansion (BE) lenses 1352, transmissive grating 1354, focusing lens 1356, field lenses 1358, and OCT sensor 1360. In some embodiments, BE lenses 1352 may be configured to expand light (e.g., from the subject's pupil) received via beam splitter 1322. In some embodiments, transmissive grating 1354 may be configured to increase symmetry and reduce aberrations in light received via beam splitter 1322. In some embodiments, transmissive grating 1354 may be configured to transmit the received light at a Littrow angle. In some embodiments transmissive grating 1354 may be configured to split the received light by wavelength. For example, transmissive grating 1354 may have a dispersion grating between 1200-1800 lines/millimeter (mm), such as between 1500-1800 lines/mm, and/or 1800 lines/mm. In some embodiments, focusing lens 1356 may be configured to focus light from transmissive grating 1354 on OCT sensor 1360 via field lenses 1358. In some embodiments, OCT detection components 1350 may also include a polarizer positioned in the path from beam splitter 1322 to OCT sensor 1360.

In some embodiments, OCT sensor 1360 may be configured to capture one or more OCT images and/or measurements using light received via BE lenses 1352, transmissive grating 1354, focusing lens 1356, and field lenses 1358. In some embodiments, OCT sensor 1360 may be configured to determine a path length difference between light received from the subject's eye via sample components 1320 and light received from reference components 1340. For example, OCT sensor 1360 may be configured to determine a phase difference between the light received from the subject's eye and from reference components 1340. In some embodiments, OCT sensor 1360 may include an interferometer such as a Mach-Zehnder interferometer and/or a Michelson interferometer. In some embodiments in which source components 1310 include multiple laser diodes, the spectrum of each laser diode may be provided and/or superimposed by transmissive grating 1354 over separate wavelengths on OCT sensor 1360.

In some embodiments, fixation components 1370 may be configured to display to the subject's eye, via fixation dichroic 1330, a visible fixation object. In FIG. 13, fixation components 1370 include fixation display 1372, fixation lenses 1374, and pupil relay 1376. In some embodiments, fixation display 1372 may be configured to provide fixation light to the subject's eye via fixation lenses to be relayed to the subject's eye (e.g., pupil) via pupil relay 1376. In some embodiments, fixation lenses 1374 may be adjustable to provide variable diopter compensation for the fixation light provided to the subject's eye. For example, diopter 1390 may be configured to adjust a positioning of fixation lenses 1374 to provide variable diopter compensation.

In some embodiments, IR components 1380 may be configured to provide and/or capture IR light to and/or from the subject's eye via IR dichroic 1328. In FIG. 13, IR components include IR LEDs 1382, MV lenses 1382, and IR camera 1386. In some embodiments, IR LEDs 1382 may be configured to provide IR light to IR dichroic 1328 to illuminate the subject's eye. In some embodiments, IR LEDs 1382 may be arranged in a ring on the surface of a mask having an aperture that permits received IR light to pass through the aperture to IR camera 1386. In some embodiments, MV lenses 1384 may be adjustable to provide variable diopter compensation for the received IR light. For example, diopter motor 1390 may be configured to adjust the positioning of MV lenses 1384 to provide variable diopter compensation. In some embodiments, IR camera 1386 may be configured to capture one or more IR images and/or measurements using IR light received via MV lenses 1384.

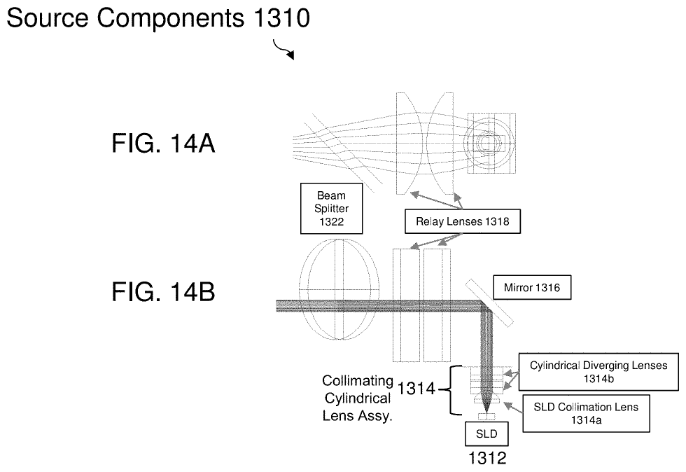

FIG. 14A is a bottom view of source components 1310 of OCT and IR components 1300, according to some embodiments. FIG. 14B is a side view of source components 1310, according to some embodiments. As shown in FIG. 14A, relay lenses 1318 may include two PCX lenses with convex surfaces facing one another. As shown in FIG. 14B, collimating cylindrical lens assembly 1314 may include a PCX SLD collimation lens 1314b and cylindrical diverging lenses 1314b. In some embodiments, mirror 1316 may be a fold-flat mirror.

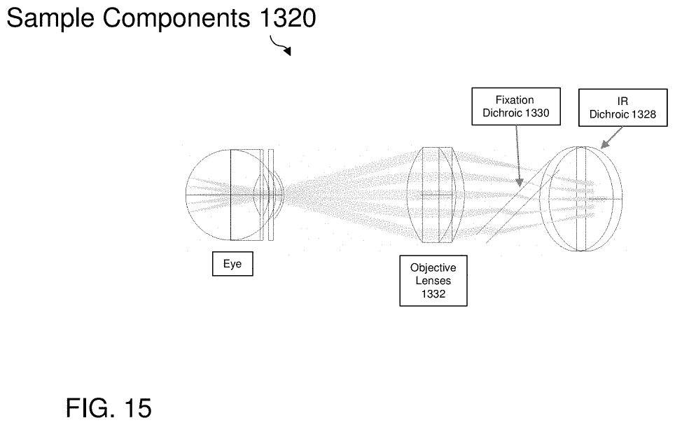

FIG. 15 is a top view of sample components 1320 of OCT and IR components 1300, according to some embodiments. As shown in FIG. 15, fixation dichroic 1330 and IR dichroic 1328 may be oriented at different angles with respect to the optical path toward the subject's eye, so as to reflect light toward the respective fixation components 1370 and IR components 1380, which may be positioned along optical paths that are perpendicular to the optical path toward the subject's eye.

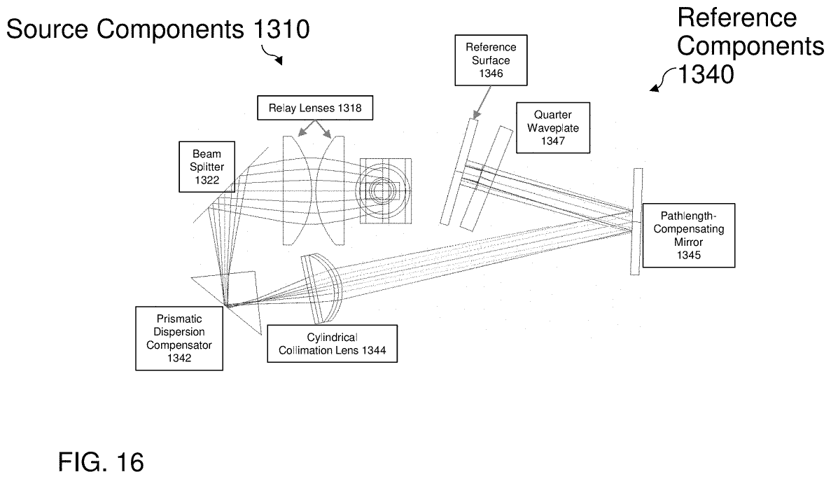

FIG. 16 is a top view of source components 1310 and reference components 1340 of OCT and IR components 1300, according to some embodiments. As shown in FIG. 16, reference components 1340 may also include a path length compensating mirror 1345 and quarter waveplate positioned between cylindrical collimation lens 1344 and reference surface 1346. It should be appreciated that, in some embodiments, prismatic dispersion compensator 1342 may include a window and a fold-flat minor.

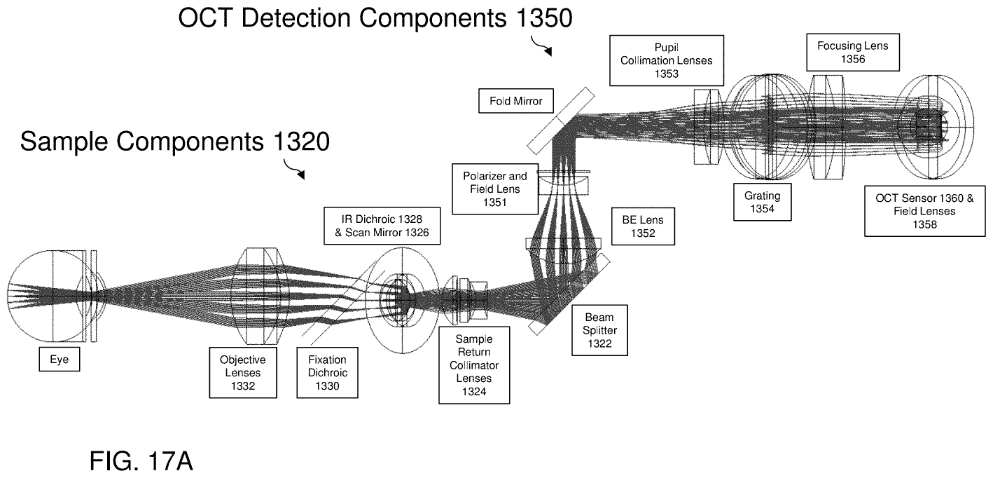

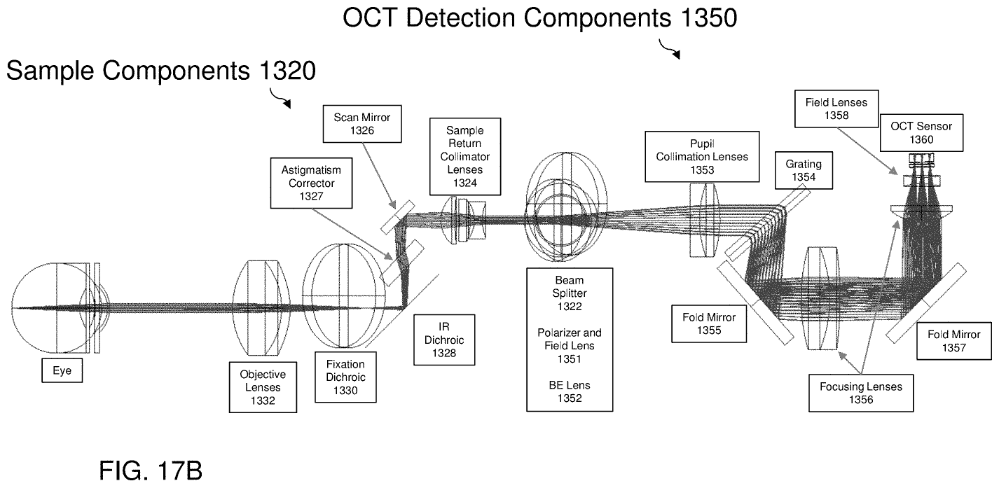

FIG. 17A is a bottom view of sample components 1320 and OCT detection components 1350 of OCT and IR components 1300, according to some embodiments. FIG. 17B is a side view of source components 1320 and OCT detection components 1350, according to some embodiments. As shown in FIG. 17A, OCT detection components 1350 may further include a polarizer and PCV field lens 135, a fold-mirror, and pupil collimating lenses 1353 positioned between BE lens 1352 and transmissive grating 1354. In FIGS. 17A-17B, pupil collimating lenses 1353 include a PCV lens and a PCX lens. As shown in FIG. 17B, sample components 1320 may further include an astigmatism corrector 1327 positioned between scan mirror 1326 and IR dichroic 1328. Also shown in FIG. 17B, focusing lenses 1356 of OCT detection components 1350 may include a PCV lens and PCX lens positioned between fold-minors 1355 and 1357, which are positioned between transmissive grating 1354 and OCT sensor 1360, and a PCX lens positioned between fold-mirror 1357 and field lenses 1358. In FIG. 17B, field lenses 1358 include a biconcave lens.

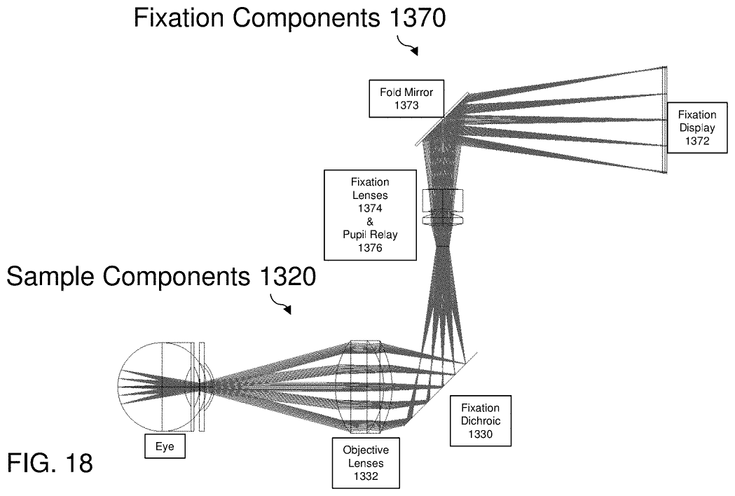

FIG. 18 is a top view of sample components 1320 and fixation components 1370 of OCT and IR components 1300, according to some embodiments. As shown in FIG. 18, fixation components 1370 may include a fold-mirror 1373 positioned between fixation lenses 1374 and fixation display 1372. In some embodiments, fixation components 1370 may include two or more fold-minors, such as positioned between fixation display 1372 and fixation lenses 1374

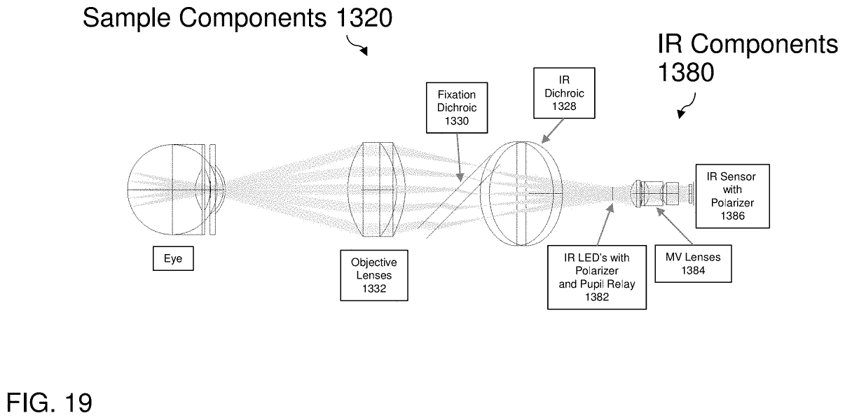

FIG. 19 is a top view of sample components 1320 and IR detection components 1380 of OCT and IR components 1300, according to some embodiments. As shown in FIG. 19, IR LEDs 1382 may include a polarizer and/or pupil relay for transmitting IR light to the subject's eye via IR dichroic 1328. In some embodiments, IR sensor 1386 may include a polarizer.

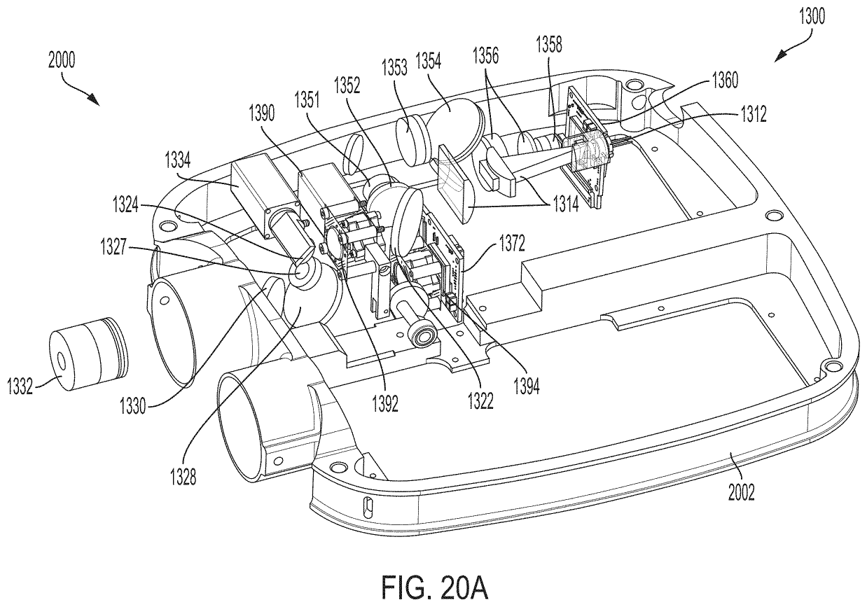

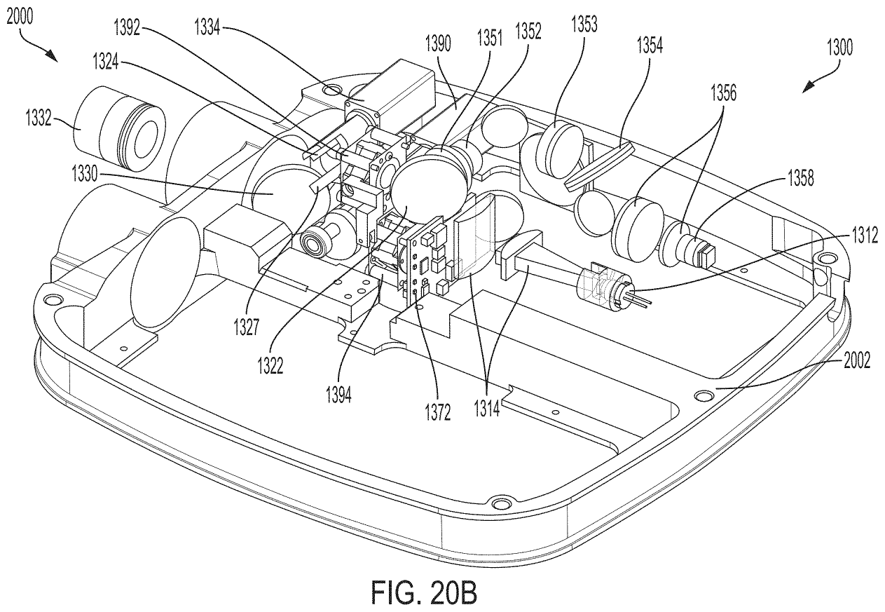

FIG. 20A is a top front perspective view of OCT and IR components 1300 positioned within an imaging and/or measuring apparatus 2000, according to some embodiments. FIG. 20B is a top rear perspective view of imaging and/or measuring apparatus 2000, according to some embodiments. As shown in FIGS. 20A-20B, OCT and IR components 1300 may be positioned within one side of a housing 2002 of imaging and/or measuring apparatus 2000. For example, white light and/or fluorescence components may be positioned in the other side of imaging and/or measuring apparatus 2000. In some embodiments, OCT and IR components 1300 may be positioned in a different section of an imaging and/or measuring apparatus and/or in separate sections.

As shown in FIGS. 20A-20B, diopter motor 1390 may be mechanically coupled to collimator flexure 1392 and diopter flexure 1394. In some embodiments, the collimator and diopter flexures may be configured in the manner described herein for diopter flexures 572, 772a, and/or 772b including in connection with FIGS. 5A-12. For example, diopter flexure 1394 may include collimator lenses 1324 of sample components 1320 and may be configured to adjust a positioning of collimator lenses 1324 when adjusted (e.g., compressed and/or decompressed) by diopter motor 1390. In this or another example, diopter flexure 1394 may include MV lenses 1384 of IR components 1380 and may be configured to adjust a positioning of MV lenses 1384 when adjusted by diopter motor 1390. Although not shown in FIGS. 20A-20B, it should be appreciated that OCT and IR components 1300 may alternatively or additionally include a diopter flexure that includes fixation lenses 1374 and is configured to adjust a positioning of fixation lenses. It should also be appreciated that OCT and IR components 1300 may include any number of diopter flexures, according to various embodiments.

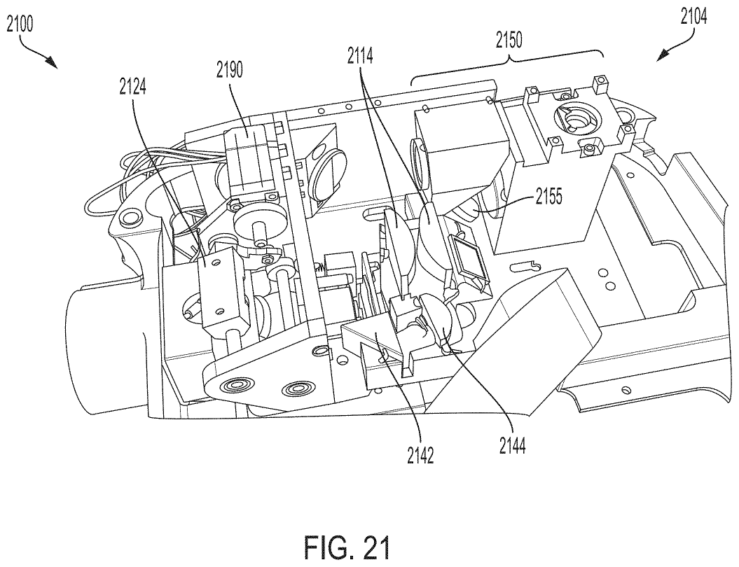

FIG. 21 is a top view of alternative exemplary OCT and IR components 2104 within an imaging and/or measuring apparatus 2100, according to some embodiments. In some embodiments, OCT and IR components 2104 may be configured in the manner described herein for OCT and IR components 1300. For example, as shown in FIG. 21, OCT and IR components 1300 include source components including collimating lenses 2114, sample components including scan mirror 2124, reference components including dispersion compensator 2142 and cylindrical collimation lens 2144, OCT detection components 2150 including mirror 2155, and diopter motor 2190.

### V. Applications

The inventors have developed improved imaging techniques that may be implemented using imaging apparatuses described herein. According to various embodiments, such imaging techniques may be used for biometric identification, health status determination, and disease diagnosis, and others.

The inventors have recognized that various health conditions may be indicated by the appearance of a person's retina fundus in one or more images captured according to techniques described herein. For example, diabetic retinopathy may be indicated by tiny bulges or micro-aneurysms protruding from the vessel walls of the smaller blood vessels, sometimes leaking fluid and blood into the retina. In addition, larger retinal vessels can begin to dilate and become irregular in diameter. Nerve fibers in the retina may begin to swell. Sometimes, the central part of the retina (macula) begins to swell, such as macular edema. Damaged blood vessels may close off, causing the growth of new, abnormal blood vessels in the retina. Glaucomatous optic neuropathy, or Glaucoma, may be indicated by thinning of the parapapillary retinal nerve fiber layer (RNFL) and optic disc cupping as a result of axonal and secondary retinal ganglion cell loss. The inventors have recognized that RNFL defects, for example indicated by OCT, are one of the earliest signs of glaucoma. In addition, age-related macular degeneration (AMD) may be indicated by the macula peeling and/or lifting, disturbances of macular pigmentation such as yellowish material under the pigment epithelial layer in the central retinal zone, and/or drusen such as macular drusen, peripheral drusen, and/or granular pattern drusen. AMD may also be indicated by geographic atrophy, such as a sharply delineated round area of hyperpigmentation, nummular atrophy, and/or subretinal fluid.

Stargardt's disease may be indicated by death of photoreceptor cells in the central portion of the retina. Macular edema may be indicated by a trench in an area surrounding the fovea. A macular hole may be indicated by a hole in the macula. Eye floaters may be indicated by non-focused optical path obscuring. Retinal detachment may be indicated by severe optic disc disruption, and/or separation from the underlying pigment epithelium. Retinal degeneration may be indicated by the deterioration of the retina. Central serous retinopathy (CSR) may be indicated by an elevation of sensory retina in the macula, and/or localized detachment from the pigment epithelium. Choroidal melanoma may be indicated by a malignant tumor derived from pigment cells initiated in the choroid. Cataracts may be indicated by opaque lens, and may also cause blurring fluorescence lifetimes and/or 2D retina fundus images. Macular telangiectasia may be indicated by a ring of fluorescence lifetimes increasing dramatically for the macula, and by smaller blood vessels degrading in and around the fovea. Alzheimer's disease and Parkinson's disease may be indicated by thinning of the RNFL. It should be appreciated that diabetic retinopathy, glaucoma, and other such conditions may lead to blindness or severe visual impairment if not properly screened and treated.

In another example, optic neuropathy, optic atrophy and/or choroidal folding can be indicated in images captured using techniques described herein. Optic neuropathy and/or optic atrophy may be caused by damage within the eye, such as glaucoma, optic neuritis, and/or papilledema, damage along the path of the optic nerve to the brain, such as a tumor, neurodegenerative disorder, and/or trauma, and/or congenital conditions such as Leber's hereditary optic atrophy (LHOA) autosomal dominant optic atrophy (ADOA). For example, compressive optic atrophy may be indicated by and/or associated with such extrinsic signs as pituitary adenoma, intracranial meningioma, aneurysms, craniopharyngioma, mucoceles, papilloma, and/or metastasis, and/or such extrinsic signs as optic nerve glioma, optic nerve sheath (ONS) meningioma, and/or lymphoma. Vascular and/or ischemic optic atrophy be indicated by and/or associated with sector disc pallor, non-arteritic anterior ischemic optic neuropathy (NAION), arteritic ischemic optic neuropathy (AION), severe optic atrophy with gliosis, giant cell arteritis, central retinal artery occlusion (CRAO), carotid artery occlusion, and/or diabetes. Neoplastic optic atrophy may be indicated by and/or associated with lymphoma, leukemia, tumor, and/or glioma Inflammatory optic atrophy may be indicated by sarcoid, systemic lupus erythematosus (SLE), Behcet's disease, demyelination, such as multiple-sclerosis (MS) and/or neuromyelitis optica spectrum disorder (NMOSD) also known as Devic disease, allergic angiitis (AN), and/or Churg-Strauss syndrome. Infectious optic atrophy may be indicated by the presence of a viral, bacterial, and/or fungal infection. Radiation optic neuropathy may also be indicated.

Moreover, in some embodiments, an imaging apparatus may be configured to detect a concussion at least in part by tracking the movement of a person's eye(s) over a sequence of images. For example, iris sensors, white light imaging components, and/or other imaging components described herein may be configured to track the movement of the person's eyes for various indications of a concussion. Toxic optic atrophy and/or nutritional optic atrophy may be indicated in association with ethambutol, amiodarone, methanol, vitamin B12 deficiency, and/or thyroid ophthalmopathy. Metabolic optic atrophy may be indicated by and/or associated with diabetes. Genetic optic atrophy may be indicated by and/or associated with ADOA and/or LHOA. Traumatic optic neuropathy may be indicated by and/or associated with trauma to the optic nerve, ONS hematoma, and/or a fracture.

Accordingly, in some embodiments, a person's predisposition to various medical conditions may be determined based on one or more images of the person's retina fundus captured according to techniques described herein. For example, if one or more of the above described signs of a particular medical condition (e.g., macula peeling and/or lifting for AMD) is detected in the captured image(s), the person may be predisposed to that medical condition.

The inventors have also recognized that some health conditions may be detected using fluorescence imaging techniques described herein. For example, macular holes may be detected using an excitation light wavelength between 340-500 nm to excite retinal pigment epithelium (RPE) and/or macular pigment in the subject's eye having a fluorescence emission wavelength of 540 nm and/or between 430-460 nm. Fluorescence from RPE may be primarily due to lipofuscin from RPE lysomes. Retinal artery occlusion may be detected using an excitation light wavelength of 445 nm to excite Flavin adenine dinucleotides (FAD), RPE, and/or nicotinamide adenine dinucleotide (NADH) in the subject's eye having a fluorescence emission wavelength between 520-570 nm. AMD in the drusen may be detected using an excitation light wavelength between 340-500 nm to excite RPE in the subject's eye having a fluorescence emission wavelength of 540 nm and/or between 430-460 nm. AMD including geographic atrophy may be detected using an excitation light wavelength of 445 nm to excite RPE and elastin in the subject's eye having a fluorescence emission wavelength between 520-570 nm. AMD of the neovascular variety may be detected by exciting the subject's choroid and/or inner retina layers. Diabetic retinopathy may be detected using an excitation light wavelength of 448 nm to excite FAD in the subject's eye having a fluorescence emission wavelength between 590-560 nm. Central serous chorio-retinopathy (CSCR) may be detected using an excitation light wavelength of 445 nm to excite RPE and elastin in the subject's eye having a fluorescence emission wavelength between 520-570 nm. Stargardt's disease may be detected using an excitation light wavelength between 340-500 nm to excite RPE in the subject's eye having a fluorescence emission wavelength of 540 nm and/or between 430-460 nm. Choroideremia may be detected using an excitation light wavelength between 340-500 nm to excite RPE in the subject's eye having a fluorescence emission wavelength of 540 nm and/or between 430-460 nm.

The inventors have also developed techniques for using a captured image of a person's retina fundus to diagnose various health issues of the person. For example, in some embodiments, any of the health conditions described above may be diagnosed.

In some embodiments, imaging techniques described herein may be used for health status determination, which may include determinations relating to cardiac health, cardiovascular disease and/or cardiovascular risk, anemia, retinal toxicity, body mass index, water weight, hydration status, muscle mass, age, smoking habits, blood oxygen levels, heart rate, white blood cell counts, red blood cell counts, and/or other such health attributes. For example, in some embodiments, a light source having a bandwidth of at least 40 nm may be configured with sufficient imaging resolution capturing red blood cells having a diameter of 6 μm and white blood cells having diameters of at least 15 μm. Accordingly, imaging techniques described herein may be configured to facilitate sorting and counting of red and white blood cells, estimating the density of each within the blood, and/or other such determinations.

In some embodiments, imaging techniques described herein may facilitate tracking of the movement of blood cells to measure blood flow rates. In some embodiments, imaging techniques described herein may facilitate tracking the width of the blood vessels, which can provide an estimate of blood pressure changes and profusion. For example, an imaging apparatus as described herein configured to resolve red and white blood cells using a 2-dimensional (2D) spatial scan completed within 1 μs may be configured to capture movement of blood cells at 1 meter per second. In some embodiments, light sources that may be included in apparatuses described herein, such as super-luminescent diodes, LEDs, and/or lasers, may be configured to emit sub-microsecond light pulses such that an image may be captured in less than one microsecond. Using spectral scan techniques described herein, an entire cross section of a scanned line (e.g., in the lateral direction) versus depth can be captured in a sub-microsecond. In some embodiments, a 2-dimensional (2D) sensor described herein may be configured to capture such images for internal or external reading at a slow rate and subsequent analysis. In some embodiments, a 3D sensor may be used. Embodiments described below overcome the challenges of obtaining multiple high quality scans within a single microsecond.

In some embodiments, imaging apparatuses described herein may be configured to scan a line aligned along a blood vessel direction. For example, the scan may be rotated and positioned after identifying a blood vessel configuration of the subject's retina fundus and selecting a larger vessel for observation. In some embodiments, a blood vessel that is small and only allows one cell to transit the vessel in sequence may be selected such that the selected vessel fits within a single scan line. In some embodiments, limiting the target imaging area to a smaller section of the subject's eye may reduce the collection area for the imaging sensor. In some embodiments, using a portion of the imaging sensor facilitates increasing the imaging frame rate to 10 s of KHz. In some embodiments, imaging apparatuses described herein may be configured to perform a fast scan over a small area of the subject's eye while reducing spectral spread interference. For example, each scanned line may use a different section of the imaging sensor array. Accordingly, multiple depth scans may be captured at the same time, where each scan is captured by a respective portion of the imaging sensor array. In some embodiments, each scan may be magnified to result in wider spacing on the imaging sensor array, such as wider than the dispersed spectrum, so that each depth scan may be measured independently.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor, but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

The acts performed as part of the methods may be ordered in any suitable way.

Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The terms “front” and “rear,” used herein in the context of describing the exemplary imaging and/or measuring apparatuses and portions thereof shown in the drawings, refer to portions of the imaging and/or measuring apparatus facing and/or positioned proximate the subject to be imaged and facing and/or positioned opposite from the subject to be imaged, respectively. It should be appreciated that imaging and/or measuring apparatuses could take other forms in which elements or views described herein as “front” or “rear” may other directions or be positioned differently with respect to the subject or subjects to be imaged, as embodiments described herein are not so limited.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

What is claimed is:

1. An imaging and/or measuring device comprising an adjustable flexure having one or more lenses therein.

2. The imaging and/or measuring device of claim 1, wherein the adjustable flexure is configured to adjust the positioning of the one or more lenses when the adjustable flexure is compressed and/or decompressed.

3. The imaging and/or measuring device of claim 2, wherein the adjustable flexure comprises an aperture having the one or more lenses therein, wherein the adjustable flexure is configured to be compressed and/or decompressed in a first direction parallel to a second direction through the aperture.

4. The imaging and/or measuring device of claim 3, wherein the adjustable flexure comprises:
an exterior portion;
an interior portion comprising the aperture; and
a flex portion flexibly coupling the exterior portion to the interior portion and configured to allow the interior and exterior portions to move, in the first direction, relative to one another.

5. The imaging and/or measuring device of claim 1, further comprising:
an objective lens; and
an imaging and/or measuring sensor,
wherein the adjustable flexure is positioned between the objective lens and the imaging and/or measuring sensor.

6. The imaging and/or measuring device of claim 5, wherein the imaging and/or measuring sensor includes at least one member selected from the group consisting of:
a white light imaging and/or measuring sensor;
a fluorescence imaging and/or measuring sensor;
an optical coherence tomography (OCT) imaging and/or measuring sensor; and
an infrared (IR) imaging and/or measuring sensor.

7. The imaging and/or measuring device of claim 1, wherein the adjustable flexure is positioned between an objective lens of the imaging and/or measuring device and a fixation display of the imaging and/or measuring device.

8. An imaging and/or measuring apparatus, comprising:
an imaging and/or measuring device comprising an adjustable flexure configured to provide variable diopter compensation for the imaging and/or measuring device; and
a motor configured to adjust the adjustable flexure.

9. The imaging and/or measuring apparatus of claim 8, further comprising a flexure assembly comprising the adjustable flexure and a lever mechanically coupled to the adjustable flexure, wherein the motor is configured to adjust the adjustable flexure via the lever.

10. The imaging and/or measuring apparatus of claim 8, wherein the motor is configured to adjust the adjustable flexure with a precision of less than 100 micrometers.

11. The imaging and/or measuring apparatus of claim 8, wherein the imaging and/or measuring device comprises a plurality of adjustable flexures, and wherein the motor is configured to mechanically adjust, along a first direction, a first adjustable flexure of the plurality of adjustable flexures and, along a second direction that is perpendicular to the first direction, a second adjustable flexure of the plurality of adjustable flexures.

12. The imaging and/or measuring apparatus of claim 11, wherein the first adjustable flexure is configured to provide variable diopter compensation for a first imaging and/or measuring device of the imaging and/or measuring apparatus and the second adjustable flexure is configured to provide variable diopter compensation for a second imaging and/or measuring device of the imaging and/or measuring apparatus.

13. The imaging and/or measuring apparatus of claim 12, wherein the motor is further configured to mechanically adjust a third adjustable flexure of the plurality of adjustable flexures along a third direction that is perpendicular to each of the first and second directions.

14. The imaging and/or measuring apparatus of claim 13, further comprising a fixation display configured to display a visible fixation object, wherein the third adjustable flexure is configured to provide variable diopter compensation for the fixation display.

15. A method comprising providing variable diopter compensation for imaging and/or measuring a person's eye using an adjustable flexure.

16. The method of claim 15, wherein the adjustable flexure comprises one or more lenses therein, and wherein providing the variable diopter compensation comprises compressing and/or decompressing the adjustable flexure to adjust a positioning of the one or more lenses.

17. The method of claim 16, further comprising adjusting the adjustable flexure using a motor.

18. The method of claim 17, further comprising mechanically adjusting, along a first direction, a first adjustable flexure of the plurality of adjustable flexures and, along a second direction that is perpendicular to the first direction, a second adjustable flexure of the plurality of adjustable flexures.

19. The method of claim 18, further comprising providing variable diopter compensation for a first imaging and/or measuring device using the first adjustable flexure and providing variable diopter compensation for a second imaging and/or measuring device using the second adjustable flexure.

20. The method of claim 19, further comprising mechanically adjusting, along a third direction that is perpendicular to each of the first and second directions, a third adjustable flexure of the plurality of adjustable flexures.
